(19) United States
(12) Patent Application Publication
(10) Pub. No.: US 2018/0042735 A1
(43) Pub. Date: Feb. 15, 2018

(54) SYSTEM AND METHOD FOR SPINAL SURGERY UTILIZING A LOW-DIAMETER SHEATHED PORTAL SHIELDING AN OBLIQUE LATERAL APPROACH THROUGH KAMBIN'S TRIANGLE

(71) Applicant: MIS IP Holdings LLC, Denver, CO (US)

(72) Inventors: Gerald R. Schell, Bay City, MI (US); Leighton LaPierre, Thornton, CO (US); Scott Noble, Denver, CO (US); Ryan Arce, Denver, CO (US); Jeffrey Schell, Denver, CO (US)

(73) Assignee: MIS IP Holdings LLC, Denver, CO (US)

(21) Appl. No.: 15/791,241

(22) Filed: Oct. 23, 2017

Related U.S. Application Data
(63) Continuation-in-part of application No. 14/875,460, filed on Oct. 5, 2015.
(60) Provisional application No. 62/059,892, filed on Oct. 4, 2014.
(60) Provisional application No. 62/411,638.

(51) Int. Cl. A61F 2/46; A61F 2/44; A61B 17/56

(52) U.S. Cl. CPC A61F 2/4611; A61B 17/56; A61F 2/4455; A61B 2017/00022

(57) ABSTRACT

Embodiments of the present invention are directed toward a system and method for facilitating the fusion of two vertebral bodies utilizing an oblique lateral surgical trajectory. Certain embodiments disclose a method for a surgical approach into one or more interbody spaces between two vertebral bodies on a trajectory through Kambin's Triangle. Certain embodiments of the invention include a method to open a pathway into a target area between two vertebral bodies of the spine using a series of one or more dilators. Certain embodiments of the invention comprise a system for placing an expandable interbody cage between two vertebral bodies. Certain embodiments of the invention incorporate a series of instruments to deliver an expandable interbody cage.

### CROSS REFERENCE TO RELATED APPLICATIONS

The present application is a continuation-in-part of U.S. patent application Ser. No. 14/875,460 filed Oct. 5, 2015, which claims the benefit of U.S. Provisional Application No. 62/059,892 filed Oct. 4, 2014, and the present application claims the benefit of U.S. Provisional Application No. 62/411,637 filed Oct. 23, 2016 and entitled “System for Spinal Fusion Surgery Utilizing a Low-Diameter Sheathed Portal Shielding an Oblique Lateral Approach”, and U.S. Provisional Patent Application No. 62/569,746 filed Oct. 9, 2017 and entitled “Neuromonitoring Dilation System,” which are all hereby incorporated by reference in their entirety.

### BACKGROUND OF THE INVENTION

Degenerative spine conditions such as kyphosis, scoliosis, hyperlordosis, spondylolisthesis and others can lead to serious disease associated with the intervertebral disc. Related compression can cause pain, spinal instability, limited motion, and inflammation, which causes back pain. Conditions such as these are often treated by removing the disc, and fusing the two vertebrae on either side of the disc together into a single bony structure.

One primary aim of intervertebral fusion is to secure the vertebrae in place together, preventing them from moving relative to one another. The movement of one bony structure against another may lead to bone spurring which may impinge nerve structures and cause pain. Often, this creates a need for a surgeon to remove a part of the bone structure that impinges a nerve. This may occur via a laminectomy or facetectomy procedure to, for instance, decompress a nerve structure.

A problem associated with removing bony structures of the spine, however, is the reduction of the supportive bony tissue able to bear strain. By performing a procedure to fuse the bony structures of the spine together, in contrast, a much more stable solution may be provided. Some fusion procedures, however, notably trans-foraminal lumbar interbody fusion (TLIF) procedures, require a surgeon to remove bony tissue to access the interbody space for fusion bed creation and implant placement. While after fusion, such procedures can effectively treat pathology, the removal of bony supportive tissue elevates the risks to the patient if such a fusion fails. Therefore, significant problems remain to be solved in association with the widespread use of the methods and apparatuses associated with TLIFs.

Typical spinal fusion procedures begin with the steps associated with accessing the junction of at least two bodies of the spine generally separated by an interbody space. The access trajectory to the interbody space is of critical importance. Several problems derive from the typically known access trajectories associated with prior art methods of creating an access corridor to the interbody space. For instance, the surgeon's creation of a route through the soft and other tissue on or near the trajectory from the skin to the spine can cause damage to those and related tissues.

Typical spinal fusion procedures known in the art involve a discectomy step, intended to remove a diseased or inflamed disc between two vertebral bodies and prepare the disc space for fusion. A problem associated with the discectomy step, however, is that the tools and steps have heretofore not adequately been developed to accomplish an optimal discectomy via an oblique approach traversing the area of Kambin's Triangle through a tube of 10 millimeters or less. Following the discectomy step, typical spinal fusion procedures incorporate a decortication step. During the decortication, a surgeon scrapes or scratches the end plates of the vertebral bodies to prepare the fusion bed. Decortication provides access to the blood vessels that exist in the deep, cancellous bone, as well as access to the pluripotent stem cells that support the healing process. A problem associated with the decortication step, however, is that the tools and steps have heretofore not adequately been developed to accomplish an optimal decortication via an oblique approach traversing the area of Kambin's Triangle through a tube of 10 millimeters or less. Following the decortication step, a surgeon typically performs the step of deposition of bone graft material. Such bone graft material may include autograft, xenograft, allograft, and synthetic graft materials to promote fusion. The fusion process is further supported by biological factors present in the bone graft material. A problem associated with the deposition of bone graft material step, however, is that the tools and steps have heretofore not adequately been developed to accomplish an optimal deposition of bone graft material via an oblique approach traversing the area of Kambin's Triangle through a tube of 10 millimeters or less.

Common routes to access the junction and/or the associated interbody space include, for example, those established by an anterior approach during a Anterior Lumbar Interbody Fusion (“ALIF”) procedure, a posterior approach during a Posterior Lumbar Interbody Fusion (“PLIF”) procedure, a lateral approach during Lateral Lumbar Interbody Fusion (LLIF) procedure (also referred to as eXtreme Lateral Interbody Fusion “XLIF” or Direct Lateral Interbody Fusion “DLIF”) and a transforaminal approach during the previously-mentioned Trans-foraminal Interbody Fusion (“TLIF”) procedure. A variety of instruments and implants exist to facilitate fusion following these approaches.

The ALIF procedure generally approaches the spine through the front of the human body. This may require a surgeon to open the stomach with a relatively large incision (usually three to five inches), and may necessitate further cuts through soft tissue. In many cases, however, the rectus abdominus muscle and the peritoneum may be retracted to the side without further damage. A problem associated with this procedure is that the associated generally anterior path comes within the vicinity of the great vessels, which carries a risk of aortic vascular laceration and bleeding out. Once through these obstacles, one or more vertebral bodies and associated interbody spaces can then be accessed.

The PLIF procedure approaches the spine from behind, or posterior to, the vertebral bodies. In this case, another relatively large initial incision (usually three to six inches) is required. Once inside the patient's body, the surgeon strips the left and right lower back muscles off of the lamina and spinous processes at one or more vertebral levels. The lamina and spinous processes may then be removed—along with any other bone cutting that may be necessary—in order to visualize the nerves. A problem associated with this procedure is that after the nerves can be seen, the surgeon retracts them to one side, a step which carries a high incidence of nerve bruising or damage. Once the nerves are moved, the interbody space can be accessed.

The TLIF procedure, like PLIF, also begins generally posterior to the spine, but takes an off-center approach through the patient's body into the spine, rather than approaching the spine from a direct posterior angle. A problem associated with this procedure is that because of the TLIF approach angle, the surgeon is generally required to remove part of or the entire facet joint of the spine in order to visualize the vertebral bodies and interbody space and to remove the disc material. As a result of the removal of at least part of a facet, increased spinal instability can result. Accordingly, if the associated vertebral bodies do not fuse following the procedure, the patient will experience chronic instability as one side of the spine is supported by an intact facet joint while the other is not. Another problem is that in many cases, to accomplish a TLIF procedure, a surgeon must retract the dura to one side, increasing the likelihood of nerve damage.

The LLIF approach begins from a lateral position to the spine. The LLIF approach requires dissection of the oblique abdominal muscle structures and the psoas, posing risks to the patient. A problem associated with the LLIF procedure is that because this approach is performed trans-psoas, the psoas and the nerve structures therein are retracted for long periods of time, increasing to the risk of nerve damage. The resulting trauma to the psoas and sensory nerve structures may produce frequent, undesirable post-operative side effects. These effects include, but are not limited to, leg pain, numbness and foot drop.

In previously-known types of anterior Oblique Lateral Interbody Fusion surgery, also commonly referred to as the “OLIF” procedure and OLIF system offered by Medtronic (referred to herein as the “Anterior OLIF”), the surgeon utilizes an anterior oblique trajectory to the spine during surgery to avoid the psoas muscle. Further, the trajectory employed by the Anterior OLIF approach accesses the spine away from the peritoneum, which provides advantages over the ALIF approach. With the exception of the iliolumbar vein and possible transitional bifurcation of great vessels, the Anterior OLIF trajectory also avoids most vasculature. Previously-known Anterior OLIF approaches can also advantageously lower the risks of tissue damage to the paraspinal muscles, nerve impaction to the spinal cord, epidural scarring, perineural fibrosis, and iatrogenic trauma. As a result, there is less tissue damage, and injury to the psoas muscle and lumbar plexus is avoided. Because of this, there is a much lower risk of sciatica-related neuropathies, such as cruralgia.

An alternative procedure to the above approaches is known as the Oblique Lateral Lumbar Interbody Fusion approach (referred to herein as “OLLIF” or the “OLLIF procedure” or the “OLLIF approach”), where the surgeon approaches from a posterior oblique trajectory to avoid the great vessels and also to cause minimal tissue trauma. Despite the remarkable advantages of the OLLIF, many surgeons have been reluctant to adopt the technique due to the required passage through Kambin's Triangle, which may place one or more of the exiting nerves and/or nerve roots at risk. In spinal anatomy, Kambin's Triangle is known as a generally right triangle that is defined by the exiting nerve (forming the hypotenuse), the caudal vertebral body (forming the base) and the traversing nerve root (forming the height). As used herein, the term “Kambin's Triangle” more generally refers to the area generally bounded by the exiting nerve, the vertebral body and the traversing nerve root, though the structures forming the boundary may not truly resemble a triangle, and though the boundary may not form a closed, contiguous loop.

A major problem associated with OLLIF is the trajectory near the nerves forming the boundary of Kambin's Triangle. In previously-known OLLIF methods and systems, without protection against impacting the nerves of Kambin's Triangle, a high incidence of associated nerve bruising or other nerve trauma has been known. Prior art solutions utilizing the OLLIF approach have not yet solved the challenges associated with establishing a durable trajectory for passage of implantation and implants through a shielded approach with a sufficiently small diameter to enable passage through Kambin's Triangle, protecting such implantation and implants from harming the nerves associated with Kambin's Triangle. A related problem associated with the approach stems from the diminutive dimensions of Kambin's Triangle. Generally, the diameter of space available to create a path directly through Kambin's Triangle is 15 millimeters or less. Therefore, the optimal implants and instrumentation designed to traverse Kambin's Triangle and accomplish a successful fusion procedure with a sufficiently low diameter remain to be developed. There is a need for an implant design, and a corresponding design for a system of surgical instrumentation, to enable spinal fusion surgery with the placement of an implant customized to fit through Kambin's Triangle to enable the avoidance damage to the structures comprising or near Kambin's Triangle.

Unlike the TLIF procedure, in an OLLIF procedure bony structures (for instance, the bony structures comprising the facet joint) do not need to be removed, which maximizes spinal stability during healing post-procedure. As the pathway is relatively avascular and less innervated, previously-known OLLIF approaches lower the risk of complication during discectomy and end plate preparation. As many as 3 or more levels of fusion can be performed in this manner, through a small, 4 cm incision. Still, many surgeons prefer the more ubiquitous TLIF procedure, as it allows surgeons to avoid the less familiar and more clustered nerves associated with Kambin's Triangle. Therefore, a need remains to develop instrumentation and implants associated with an enhanced OLLIF procedure that more safely allows surgeons to traverse the anatomy near the trajectory associated with the OLLIF surgical approach.

An advantage of the OLLIF procedure over the LLIF procedures in particular is the comparatively lower amount of blood loss during surgery. Previously-known OLLIF approaches also tend to have a lower incidence of hernias and ileuses than LLIF. Unlike the LLIF approach, typical previously-known OLLIF procedures avoid the psoas muscle. As such, with previously-known OLLIF approaches, there is a reduced incidence of nerve trauma associated with nerves in or near the psoas compared to LLIF and other approaches that require a trans-psoas access. Still, many surgeons prefer the better known LLIF procedure, as it allows surgeons to avoid the less familiar and more clustered nerves associated with Kambin's Triangle. Further, the relatively smaller footprint of implants traditionally associated with OLLIF may lead to a higher risk of subsidence relative to the LLIF procedure. Therefore, a need remains to develop instrumentation and implants associated with an enhanced OLLIF procedure that more safely allows surgeons to traverse the anatomy near the trajectory associated with the OLLIF surgical approach. There is also a need to reduce the risk of subsidence associated with implants of a diameter that can safely travel through Kambin's Triangle.

Kambin's Triangle is known to be a safe portal for epidural injection needles as such needles have a small diameter. A problem with the approach associated with prior procedures is that the dimensions of Kambin's Triangle allow for an approach trajectory path that is too narrow for many standard surgical instruments. Despite being a potentially preferable approach to the spine, many surgeons are reluctant to utilize an approach near or through Kambin's Triangle to accomplish procedures related to the interbody space because instruments and/or implants are larger than those utilized during epidural injection-type procedures, and therefore pose an increased risk of contact nerves comprising or near to Kambin's Triangle.

Moreover, a substantially lateral passage through the ilium, such as that described in U.S. Pat. No. 8,790,406 to Smith (the “'406 patent”) has yet to be perfected. More specifically, a direct lateral trajectory wide enough to access the L5-S1 interbody space for placement of an interbody cage, especially a monolithic, non-expandable cage, has led to a high incidence of intractable pain. A trajectory that traverses the ilium, but then travels above the Sacral Ala may lead to unintended deflection of instrumentation superiorly and possibly into the nerve root, causing damage to the nerves. Previously known trajectories near the Sacral Ala have failed to anticipate the need to incorporate sheathing into the surgical approach to shield structures external to the approach trajectory from the passage of instrumentation and/or implants prior to and/or during the traversal through the bone. A need therefore remains for an improved approach and cage design to enable spinal fusion at the lumbosacral (L5-S1) junction.

The geometries and anatomical structures close to the L5-S1 junction pose extreme and unique challenges related to surgical access. It is difficult, even for those skilled in the art, to comprehend the complex anatomy and multiple geometries of the sacrum, ilium and associated nerves at the L5-S1 junction. The plane of the endplate inferior to the L5-S1 disc space angles inferiorly in an anterior direction relative to the plane of the endplate superior to the L5-S1 disc space. Many fail to clearly comprehend that the structure of the sacrum partially surrounds the disc space in a lateral direction. Specifically, the Sacral Ala often extends superiorly relative to the L5-S1 inferior endplate laterally from the disc space. The Sacral Ala exists in a generally superior orientation lateral to the L5-S1 disc space relative to the lower endplate of the L5-S1 disc space. As such, at L5-S1, other approaches, including that described in U.S. Pat. No. 8,790,406 to Smith fail to appreciate and address of the location of the Sacral Ala relative to the lower ½ of Kambin's Triangle, which represents a safer “safe zone” for surgical approach (differing from the larger “safe zone” described in U.S. Pat. No. 8,790,406 to Smith) of surgical access. Moreover, other approaches including that described in U.S. Pat. No. 8,790,406 to Smith fail to incorporate steps to target the lower half of Kambin's Triangle at L5-51. As other approaches have failed to consider, the lower half of Kambin's Triangle is medial to the Sacral Ala.

The complex geometry of the sacrum, ilium and nerves near the L5-S1 junction is difficult to visualize and comprehend in two dimensions, which has contributed to the development of sub-optimal methods of surgical approach. Instead, other approaches (including that described in U.S. Pat. No. 8,790,406 to Smith) traverse through the ilium only to avoid penetrating the exterior of the Sacral Ala by traveling on a path located superior to the Sacral Ala. As such, this approach located superior to the Sacral Ala takes a path closer to, or in contact with, the nerve root exiting L5, thereby causing risk.

Previously known approaches travelling superior to the Sacral Ala travel closer to the L5 nerve root, which forms a boundary of Kambin's Triangle. Thus, such previously known approaches targeting the upper half of Kambin's Triangle place the L5 nerve root at risk. A problem associated with the methods associated with previously known approaches is that the instrumentation and implants following such methods often brush off and are forced in a superior direction by the exterior surface of the Sacral Ala, resulting in a dangerous and undesirable method of surgical approach leading to the risk of damaging or contacting the L5 nerve root. Therefore, a need exists for a different method and system to surgically approach the L5-S1 disc space to avoid damage to the L5 nerve root and to ensure patient safety.

### BRIEF SUMMARY OF CERTAIN EMBODIMENTS OF THE INVENTION

The certain embodiments described herein are preferable, in many cases, to the other approaches presented above. The certain embodiments described involves accessing the interbody space or the vertebral bodies from a posterior-oblique lateral trajectory, it is not necessary to retract the dura as with the PLIF or TLIF approaches, which thereby lowers the risk of nerve damage relative to those approaches.

Embodiments of the present invention are directed toward improvements in the system and method for facilitating the fusion of two vertebral bodies utilizing an oblique lateral surgical trajectory. Certain embodiments of the invention accesses the interbody space through a posterior-oblique lateral trajectory, which lowers the risk of nerve damage compared to other approaches such as PLIF or TLIF. Certain embodiments disclose a method for a surgical approach into one or more interbody spaces between two vertebral bodies on a trajectory through Kambin's Triangle.

Certain embodiments of the invention include a method to open a pathway into a target area between two vertebral bodies of the spine using a series of one or more dilators. Certain embodiments also incorporate a sheath, which may or may not form part of the dilation mechanism.

Certain embodiments of the invention comprise a system for placing an implant between two vertebral bodies. Certain embodiments of the invention comprise a system for placing an expandable interbody cage between two vertebral bodies. Certain embodiments of the invention incorporate a series of implant components that are assembled between two vertebral bodies. In certain embodiments of the invention an implant is defined as an expandable interbody cage comprising multiple components, including monolithic components and/or components comprising multiple parts that are individually placed through a sheath into an interbody space prior to combining the components into a fully linked construct, partially linked construct or loose construct comprised of implant components merely making contact with one another within the interbody space. In certain embodiments of the invention, the term “expanding the implant” refers to merely placing multiple implant components adjacent to or near one another within an interbody space, where the multiple implant components may optionally comprise monolithic implant components or implant components having multiple parts.

Certain embodiments of the invention incorporate a series of instruments to deliver an expandable interbody cage. In certain embodiments of the system the series of instruments includes a sheath. In a certain embodiment, the sheath is configured to dimensions to define an approach portal through Kambin's Triangle while protecting the structures comprising portions of Kambin's Triangle from anything passed through the sheath. Certain embodiments include an expandable interbody cage that collapses to a transit configuration that is able to travel through a sheath. In certain embodiments, an expandable interbody cage is removably attached to a guiding implement.

In certain embodiments, an implant, such as an expandable interbody cage, is provided. Certain embodiments comprise an implant having a collapsed form of a diameter size small enough during transit to traverse through a sheath. In certain embodiments, a sheath has an internal diameter of 10 millimeters or less. In certain embodiments, the implant can expand upon or after placement between two vertebral bodies following successful navigation through a sheath. In certain embodiments, an implant has features that rotate. In certain embodiments, a transit configuration or a retracted configuration indicates a form of the implant that allows passage through a sheath. In certain embodiments, a deployed configuration or an expanded configuration indicates a form of the implant that supports the vertebral disc space. In certain embodiments, a user controls the degree to which an implant switches between a transit configuration and a deployed configuration. In certain embodiments, the implant is structurally durable enough to withstand the forces necessary to physically separate two vertebrae. In certain embodiments, the implant comprises titanium, polyetheretherketone (PEEK), carbon fiber, ceramic, or other materials commonly utilized within orthopedic implants, or combinations thereof. In certain embodiments, the implant is detachably connected to delivery instruments utilized to transit the implant through the sheath to a target point between two vertebral bodies. In certain embodiments, the connection of the implant to delivery instruments is made via threaded connection points. In certain embodiments, the implant can be collapsed after placement and subsequently removed through the sheath. Certain embodiments incorporate a method to deliver and a method to remove the implant via for an oblique lateral approach through Kambin's Triangle. Certain embodiments of the invention comprise a deployment tool. In certain embodiments, the present inventors intend for the deployment tool to facilitate the placement and expansion of the implant apparatus. Certain embodiments include positioning tools, which enable a surgeon to place one or more implant at a targeted point between vertebral bodies in a desired configuration. In certain embodiments, a deployment tool is incorporated within an inserter. In certain embodiments, a deployment tool places force upon the implant apparatus, which translates from an axial dimension to one or more vertical and/or horizontal dimensions by the mechanisms incorporated within the implant. In certain embodiments, the placement of force by the inserter transforms the implant from its generally horizontal transit configuration into a deployed configuration.

In certain embodiments, the delivery tools including the deployment tool and inserter are detachable, and can therefore be removed once the implant is in position and successfully deployed.

### BRIEF DESCRIPTION OF THE DRAWINGS

FIG. 1. Illustration of an exemplary Kambin's Triangle.

FIG. 2A. A cross-sectional view of a first dilator in certain embodiments.

FIG. 2B. A cross-sectional view of a first dilator in certain embodiments.

FIG. 3A. A side view of a first dilator in certain embodiments.

FIG. 3B. A close up view of a first dilator distal end in certain embodiments.

FIG. 3C. A close up view of a first dilator distal end in certain embodiments.

FIG. 3D. A close up view of a first dilator distal end in certain embodiments.

FIG. 3E. A close up view of a first dilator distal end in certain embodiments.

FIG. 4. A bottom view of first dilator in certain embodiments.

FIG. 5. A perspective view of a first dilator in certain embodiments.

FIG. 6. A perspective view of a first dilator in certain embodiments.

FIG. 7. A perspective view of second dilator in certain embodiments.

FIG. 8. A close-up view of a distal end of a second dilator in certain embodiments.

FIG. 9. A close-up view of a proximal end of a second dilator in certain embodiments.

FIG. 10. A perspective view of a sheath in certain embodiments.

FIG. 11. A superior view of a sheath in certain embodiments.

FIG. 12. A perspective view of a dilator assembly in certain embodiments.

FIG. 13. A perspective view of a dilator assembly in certain embodiments.

FIG. 14. A perspective view of an implant in certain embodiments.

FIG. 15. A center link in certain embodiments.

FIG. 16. A center link in certain embodiments.

FIG. 17. A perspective view of an end link lower portion in certain embodiments.

FIG. 18. A perspective view of an end link upper portion in certain embodiments.

FIG. 19. A close-up view of a hinge between a center link and end link in certain embodiments.

FIG. 20. A perspective view of two end links and a dowel assembly in certain embodiments.

FIG. 21. A view of an underside of an end link in certain embodiments.

FIG. 22. A view of two end links fitting into complementary positions in certain embodiments.

FIG. 23. A perspective view of two end links and a dowel assembled in transit form in certain embodiments.

FIG. 24. A perspective view of an internal rod and two end links in a deployed form in certain embodiments.

FIG. 25. A view demonstrating the placement of an internal rod within the space between two mated end links in a deployed form, in certain embodiments.

FIG. 26. A view of a hinge between a center link and an end link, in certain embodiments.

FIG. 27. A perspective view of a dowel positioned into an assembly having two center links and four end links, in certain embodiments.

FIG. 28. An assembly of an implant in a deployed form, in certain embodiments.

FIG. 29. An implant in a transit form, in certain embodiments.

FIG. 30. A perspective view of an implant in certain embodiments.

FIG. 31. A deployment tool used with an implant in certain embodiments.

FIG. 32. An implant in certain embodiments.

FIG. 33. A diagram of steps used in the delivery of an implant in certain embodiments.

FIG. 34. A side view of an implant in certain embodiments.

FIG. 35. A cut away view of a sheath in position through Kambin's Triangle, demonstrating safe passage of implant in through a route in certain embodiments.

FIG. 36A. An implant in an interbody space in certain embodiments.

FIG. 36B. A top-down view of an implant in an interbody space in certain embodiments.

FIG. 37A. A close up view of a cutter assembly distal end in certain embodiments.

FIG. 37B. A close up view of a cutter assembly distal end in certain embodiments.

FIG. 37C. A close up view of a cutter assembly distal end in certain embodiments.

FIG. 37D. A close up view of a cutter assembly distal end in certain embodiments.

FIG. 37E. A close up view of a cutter assembly distal end in certain embodiments.

FIG. 37F. A side view of a cutter assembly with a cutter adjuster in a closed position in certain embodiments.

FIG. 37G. A side view of a cutter assembly with a cutter adjuster in an open position in certain embodiments.

FIG. 37H. A perspective view of a cutter adjuster in certain embodiments.

FIG. 37I. A perspective view of a first knob in certain embodiments.

FIG. 37J. A cross-sectional view of a knob of a cutter adjuster in certain embodiments, where a cross-section is taken from an exemplary knob, in certain embodiments.

FIG. 37K. A close-up view of a cutter assembly in certain embodiments.

FIG. 38A. A perspective view of a discectomy instrumentation in a retracted configuration in certain embodiments.

FIG. 38B. A perspective view of a discectomy instrumentation in an expanded configuration in certain embodiments.

FIG. 38C. A perspective view of a discectomy instrumentation in an expanded configuration in certain embodiments.

FIG. 39A. A perspective view of an access dilator assembly in certain embodiments.

FIG. 39B. A perspective view of a first dilator in certain embodiments.

FIG. 39C. A perspective view of a first dilator in certain embodiments.

FIG. 39D. A side view of a first dilator in certain embodiments.

FIG. 39E. A side cross-sectional view of a first dilator in certain embodiments.

FIG. 39F. A side view of a sheath in certain embodiments.

FIG. 39G. A side cross-sectional view of a sheath in certain embodiments.

FIG. 39H. A perspective view of a sheath in certain embodiments.

FIG. 39I. A side view of a first dilator in certain embodiments.

FIG. 39J. A close up sectional view of a first dilator in certain embodiments.

FIG. 39K. A close up sectional view of a first dilator in certain embodiments.

FIG. 40A. A perspective view of an implant in a retracted configuration in certain embodiments.

FIG. 40B. A view from a distal end of an implant in a retracted configuration in certain embodiments.

FIG. 40C. A view from a proximal end of an implant in a retracted configuration in certain embodiments.

FIG. 40D. A side view of an implant in a retracted configuration in certain embodiments.

FIG. 40E. A side view of an implant in a retracted configuration in certain embodiments.

FIG. 40F. A perspective view of an implant in a retracted configuration in certain embodiments.

FIG. 41A. A perspective view of an implant in an expanded configuration in certain embodiments.

FIG. 41B. A view from a distal end of an implant in an expanded configuration in certain embodiments.

FIG. 41C. A view from a proximal end of an implant in an expanded configuration in certain embodiments.

FIG. 41D. A side view of an implant in an expanded configuration in certain embodiments.

FIG. 41E. A side view of an implant in an expanded configuration in certain embodiments.

FIG. 41F. A perspective view of an implant in an expanded configuration in certain embodiments.

FIG. 42A. A perspective view of an implant in a retracted configuration in certain embodiments, with certain features shown.

FIG. 42B. A perspective view of an implant in a retracted configuration in certain embodiments, with certain features shown.

FIG. 43A. A perspective view of an implant in an expanded configuration in certain embodiments, with certain features shown.

FIG. 43B. A perspective view of an implant in an expanded configuration in certain embodiments, with certain features shown.

FIG. 44A. A front view of links in certain embodiments.

FIG. 44B. A perspective view of links in certain embodiments.

FIG. 44C. A side view of links in certain embodiments.

FIG. 44D. A perspective view of an implant in an expanded configuration in certain embodiments, with certain features shown.

FIG. 45A. A perspective view of a deployment instrument in certain embodiments.

FIG. 45B. A side view of a deployment instrument in certain embodiments.

FIG. 45C. A side view of a deployment instrument in certain embodiments.

FIG. 45D. A perspective view of a deployment instrument in certain embodiments.

FIG. 45E. A perspective view of a deployment instrument in certain embodiments.

FIG. 45F. An exploded view of an assembly including a delivery sheath, locking pin, locking pin lever, and a base tool block in certain embodiments.

FIG. 45G. A perspective view of an assembly including a delivery sheath, locking pin, locking pin lever, and a base tool block in certain embodiments.

FIG. 46. A side view of a portion of a deployment instrument in certain embodiments.

FIG. 47. A close-up view of a distal end of a deployment instrument in certain embodiments.

FIG. 48. A perspective view of an implant with two or more wedges in certain embodiments.

FIG. 49A. A perspective view from a distal end of a central component in certain embodiments of the invention.

FIG. 49B. A perspective view from a proximal end of a central component in certain embodiments of the invention.

FIG. 49C. A side view of a central component and stem in certain embodiments of the invention.

FIG. 49D. A perspective view from a proximal end of a central component in certain embodiments of the invention.

FIG. 50A. A side view of a wedge in certain embodiments of the invention.

FIG. 50B. A cross sectional view of a wedge, indicated in FIG. 50A, in certain embodiments of the invention

FIG. 50C. A perspective view of a wedge in certain embodiments of the invention.

FIG. 50D. A perspective view of a wedge in certain embodiments of the invention.

FIG. 50E. A cross-sectional view of a wedge in certain embodiments of the invention

FIG. 50F. A side view of a wedge in certain embodiments of the invention.

FIG. 51. A perspective view of four wedges in certain embodiments.

FIG. 52. A perspective view of an implant with two or more wedges in certain embodiments.

FIG. 53A. Exemplary step showing placement of a wedge through a working sheath in certain embodiments of the invention.

FIG. 53B. Exemplary step showing placement of a wedge through a working sheath in certain embodiments of the invention.

FIG. 53C. Exemplary step showing placement of a wedge through a working sheath in certain embodiments of the invention.

FIG. 53D. Exemplary step showing placement of a wedge through a working sheath in certain embodiments of the invention.

FIG. 54A. A top view of a dilator in certain embodiments of the invention.

FIG. 54B. A side view of a dilator in certain embodiments of the invention.

FIG. 54C. A perspective view from a proximal end of a dilator in certain embodiments of the invention.

FIG. 54D. A perspective view from a distal end of a dilator in certain embodiments of the invention.

FIG. 54E. A profile view of a proximal end of a dilator in certain embodiments of the invention.

FIG. 54F. A profile view of a distal end of a dilator in certain embodiments of the invention.

FIG. 55A. A perspective view of a sacrum, ilia and vertebrae where the route of a passage is through the ilium and the sacral ala to the L5-S1 interbody space, in certain embodiments.

FIG. 55B. A posterior sectional view of a portion of a sacrum and vertebrae, where the route of a passage is to the L5-S1 interbody space, in certain embodiments.

FIG. 55C. An oblique view of a sacrum and vertebrae, where the route of a passage is to the L5-S1 interbody space, in certain embodiments.

### DETAILED DESCRIPTION OF THE INVENTION

Descriptions of embodiments of the present invention disclosed herein are intended to serve as examples, and may not encompass all possible embodiments. One skilled in the art will recognize that variations to the embodiments disclosed herein may be made without compromising the essence of the invention.

Certain embodiments of the present invention are directed to a system and method for a surgical procedure to accomplish lumbar interbody fusion via a sheathed posterior oblique lateral approach. Certain embodiments of the invention incorporate one or more implants, which in varying embodiments may be expandable or non-expandable, insertable to a target point between two vertebral bodies through a low-diameter sheathed passage. Certain embodiments incorporate a variety of apparatuses including instrumentation and an expandable interbody cage insertable into a human body through a low-diameter, sheathed passage. For purposes related to the preferred embodiment of the invention, the term “low-diameter” refers to having an outer diameter equal to or less than 12 millimeters. The present inventors have recognized that a low-diameter sheath may safely pass on a trajectory through the area between the structures comprising the boundaries of Kambin's Triangle without causing permanent damage to the structures comprising the boundaries of Kambin's Triangle.

In certain embodiments, the implant is configured to expand following passage through a low-diameter sheathed passage and placement between vertebral bodies. In certain embodiments, the implant comprises an expandable interbody cage configured to a size and shape to fit through a low-diameter sheathed passage prior to expansion.

Certain embodiments are further directed towards a method of inserting instrumentation needed to prepare an interbody space (as used herein, the term “interbody space” is defined as the area generally between two vertebral bodies) for fusion and of inserting an implant into an interbody space through a sheathed passage that approaches the spine via a posterior oblique lateral trajectory.

Certain embodiments of the invention include instruments and steps associated with identifying an optimal trajectory to the interbody space. In a certain embodiment, these instruments include a radiopaque trajectory planning instrument, comprising of a thin elongated wire-like body of a length to span at least the distance between the interbody space and the incision point. In certain embodiments, the radiopaque trajectory planning instrument is visible on a radiographic image created with the instrument placed within the field of the image. Certain embodiments include a surgical skin marker that places a biocompatible solution on the patient's skin and serves to mark relevant anatomy viewed from the radiographic images.

Certain embodiments of the present invention include instruments and steps associated with neuromonitoring. The present inventors have recognized that neuromonitoring enables safe passage through Kambin's Triangle. The present inventors have also specifically recognized that steps associated with neuromonitoring allow surgeons to avoid an exiting nerve by enabling targeting of the lower half of the Kambin's Triangle associated with such exiting nerve. In certain embodiments the neuromonitoring probe is monopolar and unidirectional at the distal end. It will be appreciated that certain embodiments of a neuromonitoring probe have a distal end that electrically stimulates the nerves. In certain embodiments, a dilator assembly is adapted to receive a neuromonitoring probe, to allow the neuromonitoring probe to function while the neuromonitoring probe and dilator assembly work simultaneously to expand a passage. In certain embodiments, an access dilator assembly provided is adapted to incorporate a neuromonitoring probe into the dilator assembly. In certain embodiments, an access dilator assembly includes a slot on a proximal end configured to receive a flexible probe. In certain embodiments, a first dilator is configured to receive a neuromonitoring probe.

In certain embodiments, the system incorporates a guide wire. In varying embodiments, a guide wire is optionally referred to as a “Kirschner Wire” or “K-Wire.” In embodiments of the invention, a guide wire consists of a surgical instrument comprising a long member with an outer diameter of approximately 1 millimeter to 3 millimeters. In varying embodiments, a guide wire comprises stainless steel or nitinol. In certain embodiments, a guide wire has a sharp beveled tip. In certain embodiments, particularly where the guide wire is configured to pass through bone, the guide wire incorporates a drill tip. Certain embodiments of a guide wire incorporate a rounded blunt tip as to minimize tissue trauma.

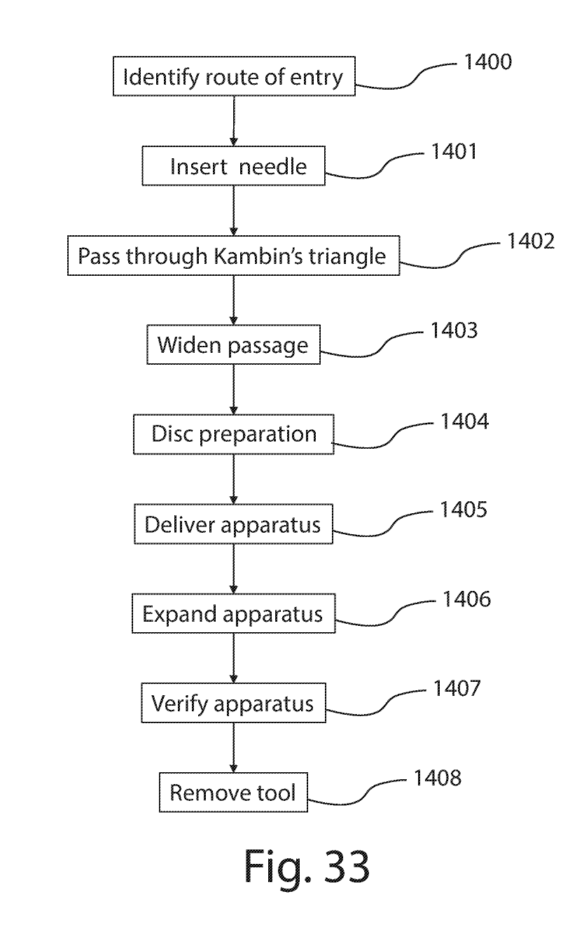

Certain embodiments of the present invention incorporate instruments and steps associated with discectomy. Discectomy may be performed during a disc preparation step 1404 as shown in FIG. 33. In certain embodiments, discectomy instrumentation comprises instruments for the removal of vertebral disc material at a targeted interbody space. In certain embodiments, discectomy instrumentation is configured to pass through a sheathed passage. In certain embodiments, discectomy instrumentation is configured to pass through a low-diameter sheathed passage in a retracted state, then partially expand within a disc space, and then return to a retracted state for removal through a low-diameter sheathed passage. In certain embodiments, discectomy instrumentation includes, for example, a disc reamer having a cylindrical hollow body, capable of accessing and fitting into the interbody space and reaming disc tissue. In certain embodiments, discectomy instrumentation includes, for example, an elongate body with the distal end having a drill bit mechanism, allowing drilling through the disc material, capturing the disc material within the grooves of the drill bit, and extracting the disc material by removing the drill bit. In certain embodiments, an endoscope may be utilized in association with discectomy instrumentation for purposes associated with the inspection of the discectomy and endplate preparation prior to and following the insertion of discectomy instrumentation.

In certain embodiments, discectomy instrumentation includes, for example, loop cutters. Loop cutters include a flat, thin, ribbon of material deployable through an elongate tube. A loop cutter is accessible in a vertebral disc space to cut the disc tissue. In certain embodiments, discectomy instrumentation may take the form of embodiments disclosed within U.S. Pat. No. 7,500,977 B2, U.S. Patent Publication No. US 2007/0260270 A1, U.S. Patent Publication No. US 2008/0033466 A1, U.S. Pat. No. 7,632,274 B2, U.S. Patent Publication No. US 2007/0265652A1, U.S. Patent Publication No. US 2005/0149034 A1, U.S. Patent Publication No. US 2003/0191474 A1, U.S. Pat. No. 7,500,972 B2, and U.S. Pat. No. 7,588,574, which are incorporated herein by reference in their entirety. In certain embodiments, the loop cutters may take the form of embodiments described within the documents referred to within the preceding sentence. In certain embodiments, the loop cutters deploy at a substantially 45 degree angle. In certain embodiments, discectomy instrumentation including cutter assemblies are configured for an oblique lateral procedure and thereby differ from previously known loop cutters in the plane of deployment into the disc space.

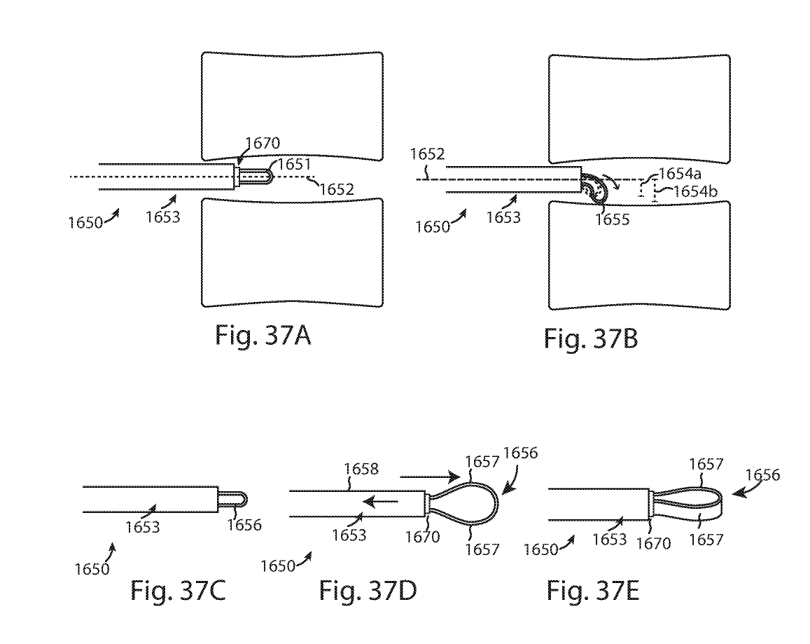

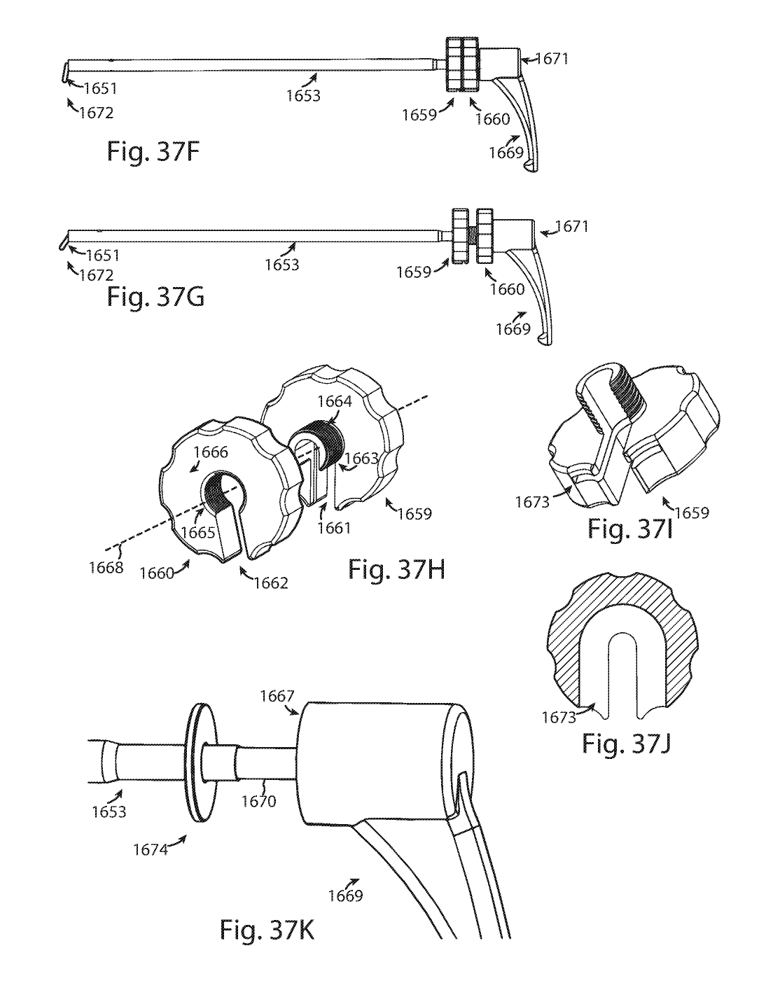

Referring to FIGS. 37A-K, in certain embodiments, a cutter assembly includes a cutter shaft, a cutter sheath, and a handle. In certain embodiments, a cutter shaft 1670 is attached to a cutter blade 1651, 1655, 1656, where a cutter sheath 1653 is concentrically placed over the cutter shaft 1670. The cutter sheath 1653, cutter shaft 1670, and handle 1669 components are preferably co-configured to enable the cutter blade and the cutter shaft 1663 to which it is attached be able to be “pushed-pulled” so as to retract the cutter blade into the cutter sheath and then extend the cutter blade from the distal end 1672 of the cutter sheath as needed.

Referring to FIG. 37A, in certain embodiments, a cutter assembly deploys a cutter blade 1651 in a plane that is parallel to the plane that intersects the longitudinal axis 1652 of the instrument in order to cut in varying heights of the disc space. In certain embodiments, cutter assembly 1650 deploys a cutter blade 1651 in a plane that is parallel to the plane of the disc space. In certain embodiments, referring to FIG. 37B, the act of sheathing the cutter blade into a protective sheath 1653 allows control of the effective radius 1654a, 1654b of the cutter blade 1655. As seen in FIG. 37B, in certain embodiments, the cutter blade is deployed generally laterally from a longitudinal axis 1652. This change in radius can be determined from the user (proximal) end of the cutter assembly to match the varying concavities and heights between the vertebral endplates, using certain embodiments of a cutter adjuster as shown in FIGS. 37F-J. In certain embodiments, the radius of a cutter blade is adjusted with a first knob 1659 and a second knob 1660. It will be appreciated that in certain embodiments, a first knob 1659 and a second knob 1660 is placed on discectomy instrumentation disclosed in U.S. Pat. No. 7,500,977 B2, U.S. Patent Publication No. US 2007/0260270 A1, U.S. Patent Publication No. US 2008/0033466 A1, U.S. Pat. No. 7,632,274 B2, U.S. Patent Publication No. US 2007/0265652A1, U.S. Patent Publication No. US 2005/0149034 A1, U.S. Patent Publication No. US 2003/0191474 A1, U.S. Pat. No. 7,500,972 B2, and U.S. Pat. No. 7,588,574, which are incorporated herein by reference in their entirety. A first knob 1659 and second knob 1660 includes a primary slot 1661, 1662 that cuts from their center axis 1668 to the outer perimeter. The primary slot allows the first and second knob to slide over the cutter shaft and/or cutter sheath. A first knob 1659 further includes a connector 1663 having threads 1664 that allows a threaded connection with a threaded opening 1665 of a second knob 1660. Referring to FIG. 37I-J, the first knob 1659 has a second slot 1673 that cuts through the mid portion of the knob 1659. It will be appreciated that a second knob 1660 includes a second slot in certain embodiments. Referring to FIG. 37K, in certain embodiments, the second slot 1673 captures an end stop 1674, which is connected to the cutter sheath 1653. In certain embodiments, an end stop is a concentric collar attached to a cutter sheath and/or cutter shaft. In order to control the distance/radius and angle of the cutter blade that is exposed at a distal end 1672, the first knob 1659 and second knob 1660 are rotated relative to each other along the threaded connection to create a distance between first and second knobs. In certain embodiments, a first surface 1666 of a second knob 1660 contacts the second surface 1667 located on a handle 1669. In certain embodiments, a cutter shaft 1670 includes an end stop, while a second knob includes a second slot. In certain embodiments, a first knob and a second knob, both having a second slot, is positioned over a cutter assembly where a cutter sheath and cutter shaft have an end stop.

In certain embodiments, the cutter is adjusted using the following steps. A first knob and second knob are threaded together so the two knobs are fully engaged. The primary slot on both knobs should be radially aligned with each other. With the cutter sheath advanced distally, where a cutter blade is fully “sheathed” or housed in the sheath in its retracted state, the cutter adjuster is placed over the cutter sheath, end stop, and cutter shaft. With the cutter adjuster attached to the cutter assembly, the cutter adjuster assembly is pulled proximally until the cutter blade is deployed. The proximally located knob (e.g. second knob) is rotated relative to the distally located knob (e.g. first knob) to retract the cutter blade into the sheath. The knobs are turned until a preferred deployment position, for example, when the distance, radius, and angle of the cutter blade is appropriate, is set. In certain embodiments, the distance, radius, and angle of the cutter blade can be adjusted in situ by rotating the first and second knobs relative to each other.

In certain embodiments, the depth of the cutter blade and angle relative to the initial approach angle allows the user to prepare the desired footprint of the interbody space during steps related to discectomy. In certain embodiments, a cutter assembly 1650 as shown in FIG. 37C-D is used. In certain embodiments, a cutter blade 1656 has side walls 1657 that extend out and spread when the cutter blade 1656 is deployed. In certain embodiments, when the cutter 1656 is deployed, the side walls 1657 extend laterally beyond the outer wall 1658 of the sheath 1653.

In certain embodiments, once the distal end of the cutter blade is in the desired location to debulk the disc space, the radius of the loop or distance of deployment may be set by the user. In certain embodiments, the cutter blade may be controllably rotated by a user using a handle affixed to the protective sheath at the proximal end to perform discectomy by removing material proximal to the superior and inferior endplates. Optionally, in certain embodiments, decortication of the superior and inferior endplates may be achieved by rotating the cutter blade to scrape the superior and inferior endplates.

In certain embodiments, discectomy instrumentation includes, for example, an endplate rasp. In certain embodiments, an endplate rasp has a spoon-shaped end, capable of accessing and fitting into the disc space, and decorticating the vertebral endplates. In certain embodiments, the discectomy instrumentation may take the form of embodiments described in U.S. Pat. No. 8,696,672 B2, U.S. Patent Publication No. 2011/0184420 A1, which are incorporated herein by reference in their entirety. In certain embodiments, the endplate rasp may take the form of embodiments described by the documents referred to within the preceding sentence. In certain embodiments, discectomy instrumentation includes, for example, disc material removal tools. Such disc material removal tools include, for example, surgical pituitaries capable of accessing and fitting into the disc space to remove disc material. In certain embodiments, the discectomy instrumentation may take the form of embodiments described in U.S. Pat. No. 8,052,613 B2, which is incorporated herein by reference in its entirety. In certain embodiments, the disc material removal tools may take the form of embodiments described by the document referred to within the previous sentence.

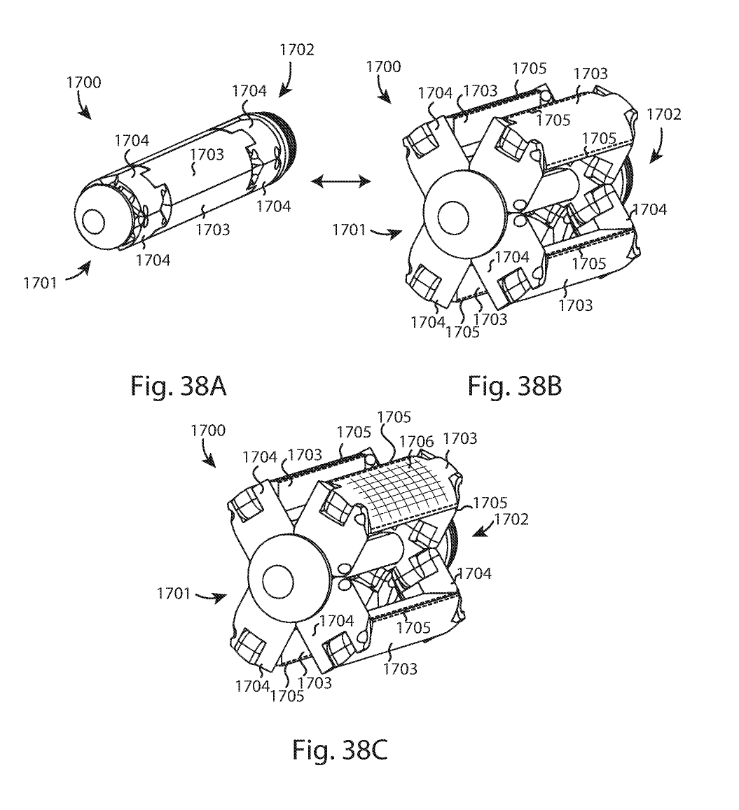

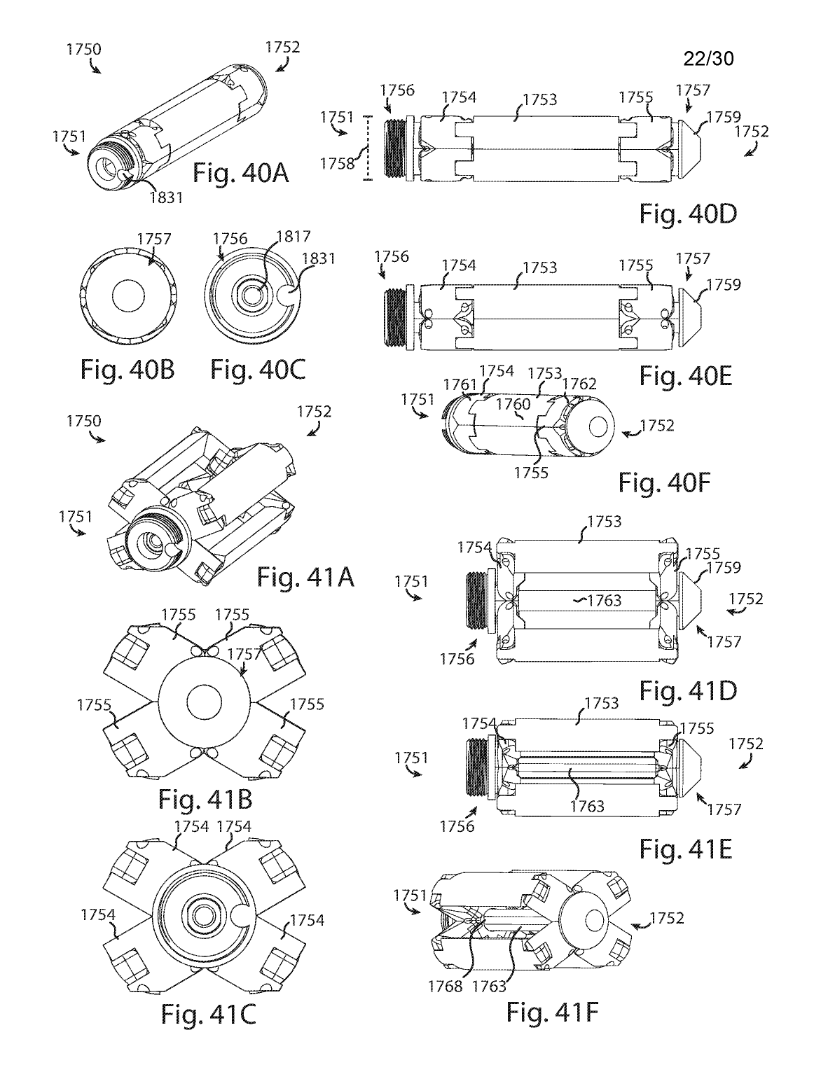

In certain embodiments, discectomy instrumentation includes, for example, an expandable discectomy tool 1700 as shown in FIG. 38A-C. In certain embodiments, the expandable discectomy tool 1700 has a distal end 1701 and a proximal end 1702 and a plurality of center links 1703 and end links 1704. In certain embodiments, the expandable discectomy tool 1700 is expanded in a similar manner to the expandable implant 1750 as exemplified and described, for example, in FIGS. 40A-F and FIGS. 41A-F. The expandable discectomy tool 1700 includes cutting edges 1705 as seen in FIG. 38B. Rotating the expandable discectomy tool 1700 in the vertebral disc space allows decortication of the upper and lower endplates. Referring to FIG. 38C, in certain embodiments, an expandable discectomy tool 1700 includes a rasping surface 1706 on one or more center links 1703.

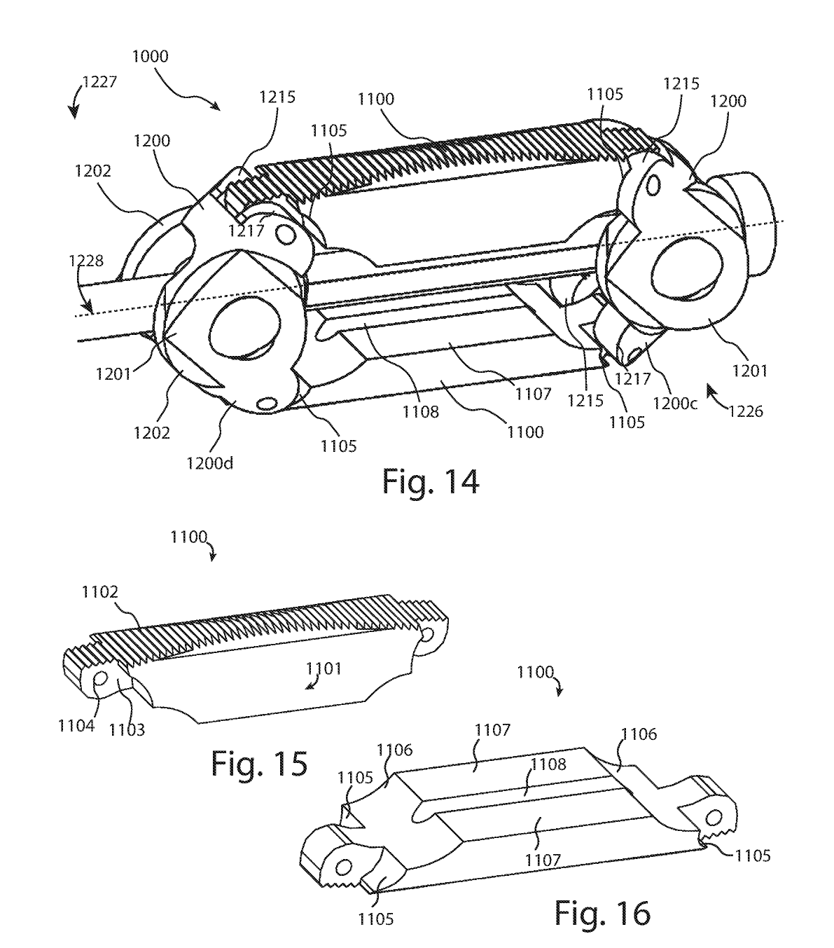

Certain embodiments of the present invention include instruments and steps associated with trialing or inserting trial instruments. It will be appreciated that a trial allows evaluation and determination of a surgical area prior to placing an implant. In certain embodiments, a trialing instrument is capable of determining the measurement of interbody space height, while simultaneously distracting two vertebrae apart from each other. The trialing instrument, and the steps associated with placing the trials are performed through a sheath. In certain embodiments the trialing instrument resembles a pituitary, comprising an elongated portion of two slidably engaged semicircular extrusions. In certain embodiments, the semicircular extrusions are then connected to a handle in such a way that upon squeezing the handle, the superior semicircular extrusion slides over the inferior semicircular extrusion. In certain embodiments, trialing instrument is performed with an expandable cage similar to those shown in FIGS. 14-32, and similar to those shown in FIGS. 40-44.

In certain embodiments, instruments and steps are adapted to safely pass one or more non-expandable implants. In certain embodiments, instruments and steps are adapted to safely pass one or more expandable interbody implants and instrumentation through a sheath into an interbody space. In certain embodiments, non-expandable interbody cages and/or expandable interbody cages, and associated instruments, are passed through a low-diameter sheathed passage. In certain embodiments, a sheath is configured to follow a passage established through Kambin's Triangle. In certain embodiments, a sheath is configured to follow a passage from the skin into a L5-S1 interbody space by first passing through an ilium, a sacroiliac joint, a sacrum, and Kambin's Triangle. In certain embodiments, a sheath is configured to follow a passage from the skin into a L5-S1 interbody space by first passing through an ilium and a sacroiliac joint, but stopping prior to the sacrum, whereby an unsheathed passage is further created through the sacrum, through Kambin's Triangle and into an interbody space. In a certain embodiment, passage through Kambin's Triangle is accomplished by shielding Kambin's Triangle from physical impact associated with the passage of instrumentation and implants by a sheath.

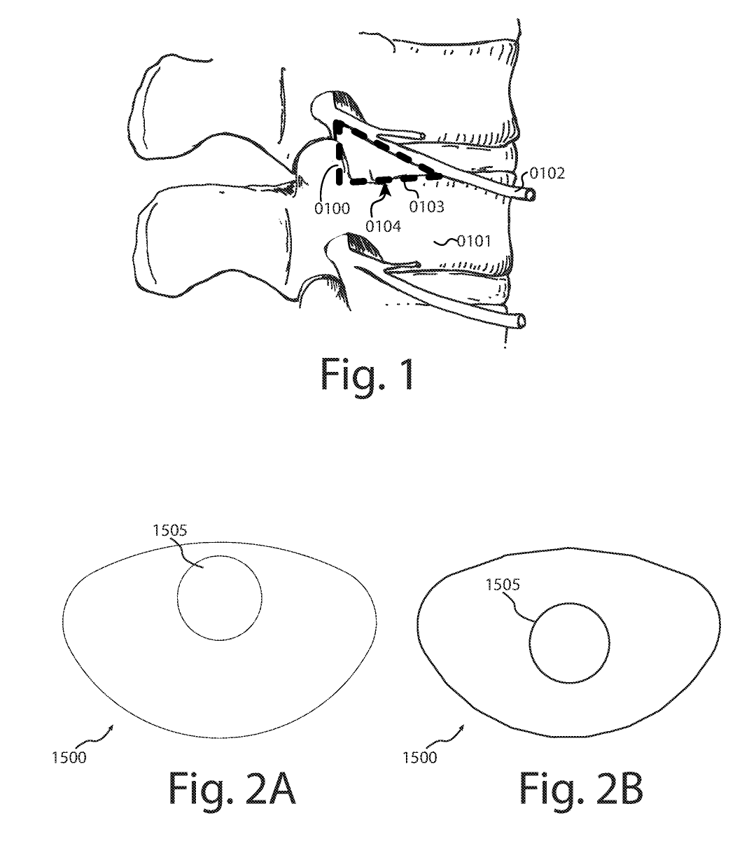

In certain embodiments, in order to safely pass through Kambin's Triangle, dilation instruments and steps are adapted to widen or dilate the passage. In one example, the passage begins at an incision point in the skin and ends within an interbody space. As seen in FIG. 1, Kambin's Triangle 0104 is defined by a traversing nerve and/or superior articular process 0100, the superior face 0103 of a vertebral body 0101, and an exiting nerve root 0102 from the superior part of the neural foramen. In certain embodiments, dilation instruments comprise surgical grade aluminum with a Type III anodized coating and/or stainless steel. In certain embodiments, such instruments comprise radiolucent properties. In certain embodiments, an endoscope may be utilized in association with instrumentation for purposes associated with the inspection of the foramen and other structures near the passage prior to and following the insertion of dilation instrumentation.

In certain embodiments, dilation instruments incorporate a tapered distal end 1530. In certain embodiments, a dilation instrument comprises a plurality of dilators. For example, a first dilator 1500 having a tapered distal end 1530 is slidably removable through a second dilator having a larger diameter. In a certain embodiment, a dilation instrument incorporates a neuromonitoring feature to allow for the detection of nerve structures located near the dilation instrument. In a certain embodiment, neuromonitoring is performed by sending an electrical current through the dilation instrument, which is measured at another point in a patient's body. In certain embodiments, the dilation instrument has a longitudinal hole to enable the dilation mechanism to slide over a prior placed neuromonitoring probe. In a certain embodiment, the series of dilators is configured such that the dimensions of the dilators can pass between the structures comprising the boundaries of Kambin's Triangle without contacting such structures while expanding the passage enough to accommodate the placement of a low-diameter sheath.

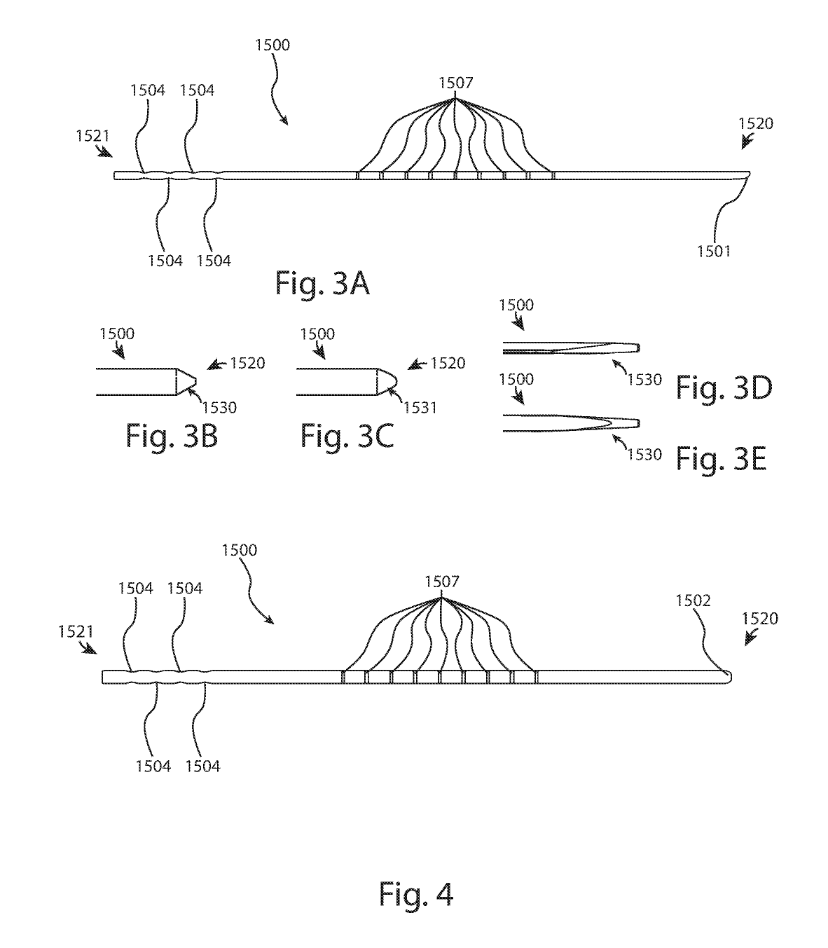

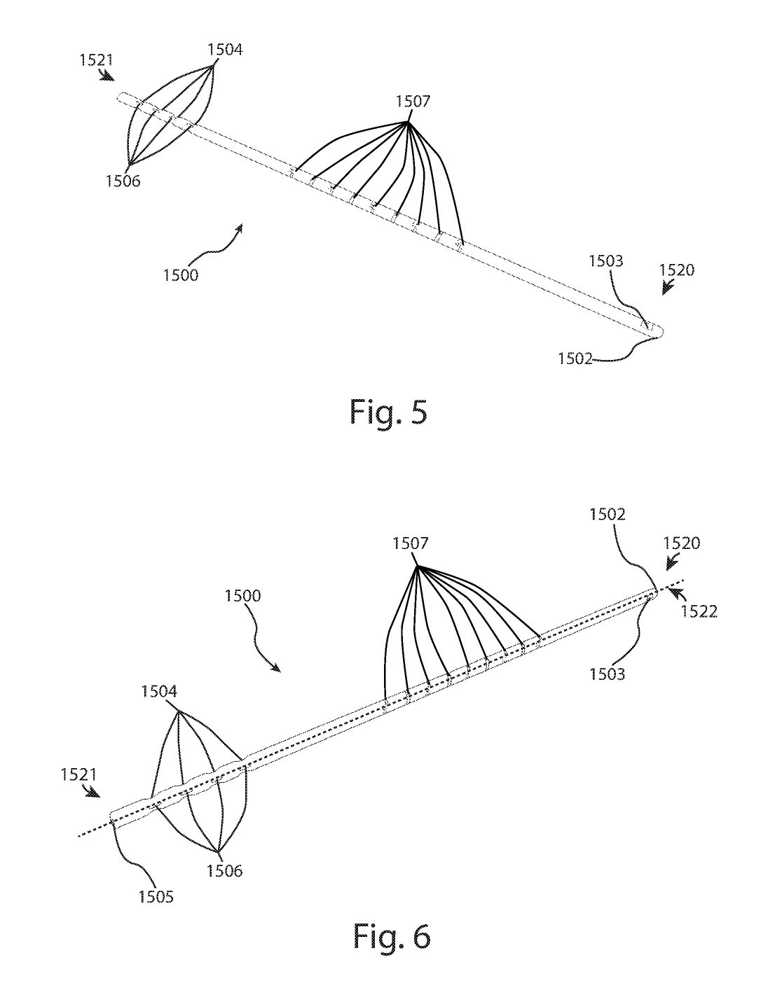

As seen in FIGS. 2-6, in certain embodiments, the dilation mechanism incorporates a first dilator 1500. In certain embodiments, a first dilator comprises a tubular extrusion with a generally oblong shaped cross-section. In certain embodiments, the first dilator 1500 is 6 millimeters wide at its widest point and 260 millimeters in length, although other sizes can be considered. Referring to FIG. 2A-B, in certain embodiments, the cross-sectional profile of the first dilator is circular, oval, or triangular. Referring to FIG. 3-6, in certain embodiment, a distal end 1520 of the first dilator 1500 comprises a bevel 1501 and rounded tip 1502. In certain embodiments, the bevel 1501 and rounded tip 1502 minimizes the occurrence of tissue disruption during passage of the first dilator through Kambin's Triangle and proximal structures. In certain embodiments, a first dilator 1500 has a circular taper, and in certain embodiments, a first dilator 1500 has a bullet-shaped tip 1531 at the distal end. Generally, an exemplary tapered end 1530 as shown in FIGS. 3B, D, and E allows for a gradual, atraumatic opening of tissue as the dilator progresses into the body. Referring to FIG. 5 and FIG. 6, in certain embodiments, the distal area of a first dilator 1500 has a reference marking 1503 used to denote which side of first dilator 1500 should orient generally superiorly, and along an exiting nerve root 0102. In certain embodiments, the proximal end 1521 of a first dilator 1500 incorporates one or more grooves 1504. In certain embodiments, the grooves 1504 are oriented in a substantially orthogonal direction relative to the longitudinal axis 1522 of the first dilator 1500. The grooves 1504 allow for improved user grip.

Referring to FIGS. 2A-B and FIG. 6, in certain embodiments, a through hole or cannula 1505 forms a contiguous channel through a first dilator 1500. In certain embodiments, the cannula 1505 exists along a longitudinal axis 1522 of the first dilator 1500. In certain embodiments, a cannula 1505 connects a proximal end 1521 and distal end 1520. In certain embodiments, a cannula 1505 is offset (see FIG. 2A), or is centered (see FIG. 2B) on a first dilator. Referring to FIG. 6, in certain embodiments, a side aperture 1506 is located within grooves 1504. A side aperture 1506 is further connected with a cannula 1505. Referring to FIG. 3, in certain embodiments, one or more depth markers 1507 are located on an outer surface of the first dilator 1500. In certain embodiments, a plurality of depth markers 1507 begin approximately 80 mm from a distal end, and ends 160 mm from a distal end, where the location of the depth marker is placed in 10 mm intervals.

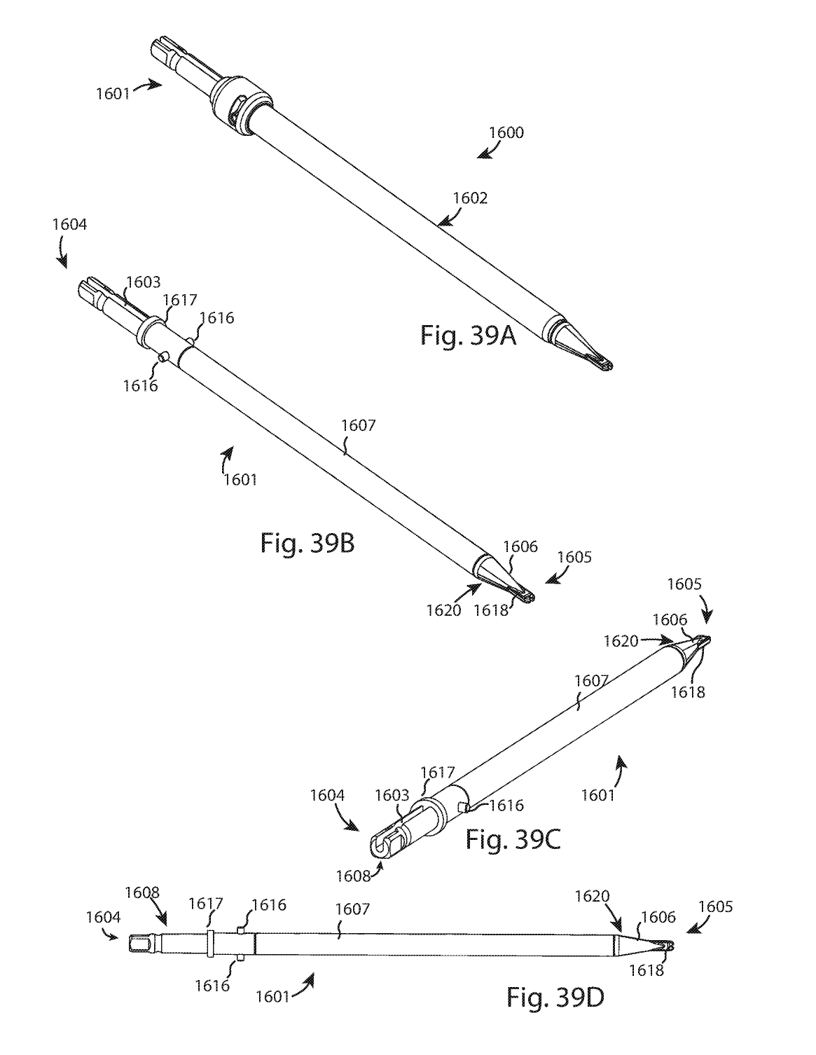

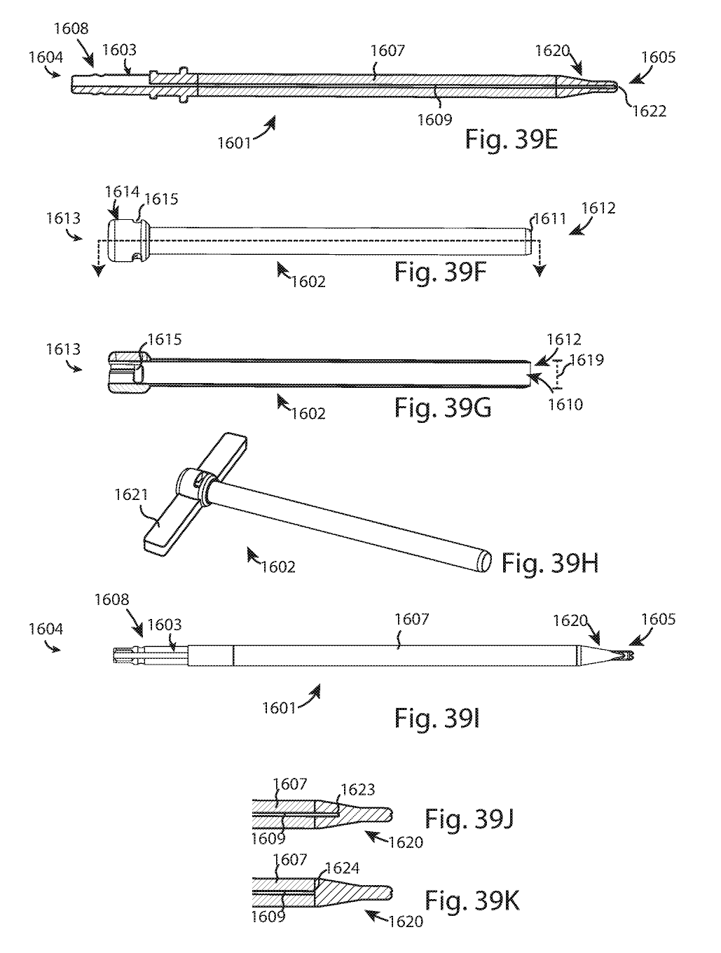

In certain embodiments, neuromonitoring occurs while dilating a pathway to a target site. In certain embodiments, an access dilator assembly 1600 as shown in FIGS. 39A-39K allows nerve monitoring, soft tissue dilation, initial disc access and the delivery of a sheath. In certain embodiments, an access dilator assembly 1600 includes a first dilator, and a sheath. In certain embodiments, a first dilator, as seen in FIGS. 39B-39E, I, is also referred to as a dilator shaft. It will be appreciated that a first dilator 1601 can be used with other instruments. In certain embodiments, neuromonitoring is performed as described in U.S. Provisional Patent Application No. 62/569,746 filed Oct. 9, 2017 and entitled “Neuromonitoring Dilation System,” which is hereby incorporated by reference in its entirety.

In certain embodiments, an access dilator assembly 1600 facilitates neuromonitoring by accommodating a standard disposable monopolar probe, such as a Cadwell 200 millimeter ball tip disposable monopolar probe, through a slot 1603 located on a proximal end 1604 of a first dilator 1601 as shown in FIGS. 39B and 39E. A standard monopolar probe may further be pushed through the cannula 1609, as seen in FIG. 39E, towards the distal end 1605. A standard disposable monopolar probe in such embodiments is delivered through the access dilator assembly 1600 prior to or during a surgical procedure. It will be appreciated that the cannula 1609 can also accommodate other instruments including guide wires or K-wires. In certain embodiments, a cannula is connected with an opening located generally near the first dilator proximal end 1604, and extends towards a first dilator distal end 1605. In certain embodiments, a cannula is connected with a tip aperture 1622 of a first dilator distal end 1605. Referring now to FIG. J-K, in certain embodiments, the cannulation is a blind hole, where the cannulation 1609 extends from the proximal end, and ends at a stop 1623 located within a volume of a distal piece 1620 that is conductive. In certain embodiments, the cannulation 1609 extends from the proximal end and ends at a stop 1624 prior to crossing into a distal piece 1620 that is conductive. In certain embodiments, the purpose of a cannulation comprising a blind hole is to allow the stimulating tip of the disposable monopolar probe to make contact with a conductive tapered tip or distal piece, and by extension allow the conductive tapered tip to have stimulation capabilities.

In certain embodiments, the distal end of the standard disposable monopolar probe is intended to make contact with an electrically conductive distal end 1605 of the first dilator 1601. In certain embodiments, the distal end 1605 of the first dilator 1601 includes a distal piece 1620 made of an electrically conductive material, such as stainless steel. In certain embodiments, the distal piece 1620 of the first dilator 1601 has a taper 1606. A tapered profile 1606 facilitates entry into the disc space and dilation up to the diameter of the sheath 1602. In certain embodiments, as seen in Figs. B-D, the distal piece 1620 of the first dilator 1601 includes a disc penetrator or flattened tip 1618. Contacting the monopolar stimulating tip of a standard disposable monopolar probe with the distal piece 1620 allows electrical stimulation of the distal end, as to determine the proximity of the access dilator assembly 1600 to nerves, including for example, edges of Kambin's Triangle.

In certain embodiments, the distal end 1605 of the access dilator assembly 1600 is electrically conductive, while the shaft 1607 is electrically insulated. In certain embodiments, the distal piece 1620 is electrically conductive. In certain embodiments, the end of a disposable monopolar probe contacts the electrically conductive distal piece 1620. The shaft 1607 has an insulating material in order to localize the electric current to the distal end 1605. The insulating quality of the shaft 1607 further prevents shunting or shorting out of the neuromonitoring signal. In certain embodiments, the insulating material of the main shaft of the dilation mechanism comprises a non-conductive metal, such as aluminum, (e.g. type III anodized aluminum). In certain embodiments, the proximal end 1604 of the access dilator assembly 1600 features a quick connect feature 1608 as seen in FIGS. 39B-D. The quick connect feature 1608 and a shaft 1607 is attached, for example, through a number of attachment mechanisms known, including, but not limited to threaded attachment, adhesive, and interference fit. It will be appreciated that a probe shaft 1607 and a distal piece 1620 are connected through a number of known attachment mechanisms.

In certain embodiments, distal end 1605 of the access dilator assembly 1600 is electrically insulated. In certain embodiments, the distal piece 1620 is electrically insulated. In such embodiment, the end of a disposable monopolar probe is exposed at the end of a distal piece 1620 through a tip aperture 1622 (shown in FIG. 39E).

Referring to FIGS. 39B-D, I, in certain embodiments, the quick connect feature 1608 allows attachment of a standard surgical handle. Referring to FIGS. 39B, 39C, 39E, and 39I, in certain embodiments, the quick connect feature 1608 and/or the shaft 1607 incorporates a slot 1603 designed to accommodate a standard disposable monopolar probe, while the standard surgical handle is attached. In certain embodiments, the slot 1603 facilitates the placement of the standard surgical handle on the quick connect feature 1608 with the monopolar probe in place by bending the standard disposable monopolar probe. Referring to FIG. 39G, in certain embodiments, the first dilator 1601 is passed through the cannulation 1610 of the probe sheath 1602. Certain embodiments of the sheath 1602 have an inner diameter 1619 of 9 mm. Referring to FIGS. 39F-G, a proximal end 1613 of the sheath 1602 includes an impact collar 1614 further having a pin slot 1615. The pin slot 1615 engages with the pin 1616 located on the first dilator 1601 (seen in FIGS. 39B-D). In certain embodiments, a sheath 1602 is assembled with a first dilator 1601 and inserted together into an interbody space. In certain embodiments, once the sheath creates a passage between an interbody space and the exterior of a patient, the first dilator 1601 is disengaged and removed. The impact collar 1614 of the sheath 1602 further contacts an impact collar 1617 located on the first dilator 1601 (seen in FIGS. 39B-D). Still referring to FIG. 39G, in certain embodiments, the distal end of the 1612 of the sheath 1602 includes a sheath bevel 1611. In certain embodiments, the bevel assists in positioning the sheath into interbody space. In certain embodiments, a sheath 1602 includes a handle 1621, as shown in FIG. 39H.

In certain embodiments, a first dilator has an outer surface lacking a pin 1616 and an impact collar 1614, as shown in FIG. 39I. In certain embodiments, a first dilator as shown in FIG. 39I allows insertion into a proximal end of a dilator or a sheath. In certain embodiments, a first dilator includes a shaft 1607 and a distal piece 1620 that are both non-conductive. In certain embodiments, a shaft 1607 and a distal piece 1620 are a unitary piece. In certain embodiments, a shaft 1607 and a distal piece 1620, and quick connect feature 1608 are non-conductive. In certain embodiments, a shaft 1607 and a distal piece 1620, and quick connect feature 1608 are a unitary piece. In certain embodiments where a distal piece 1620 is non-conductive, the monopolar stimulating tip of a standard disposable monopolar probe is exposed through the distal end 1605, through the distal piece 1620.

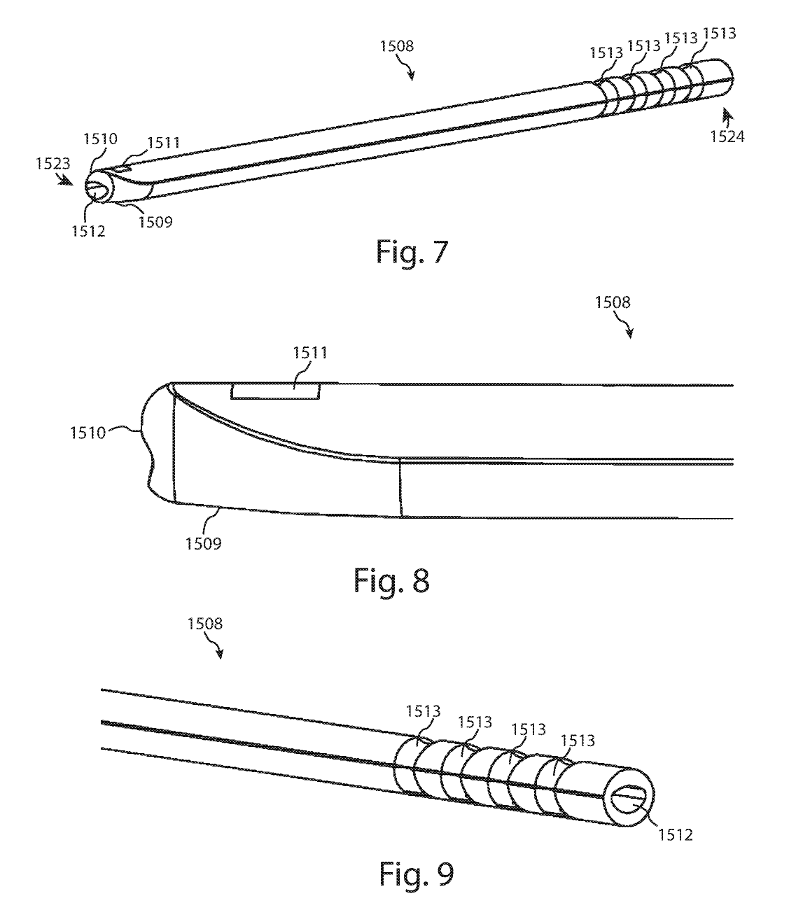

In certain embodiments, a second dilator is slidably and removably placed over a first dilator. Referring to FIGS. 7-9, a second dilator 1508 has a cross-sectional profile similarly oblong to first dilator 1500. In certain embodiments, the second dilator 1508 has an outer diameter with an 8 mm width at its widest point, and has a length of approximately 240 mm. In certain embodiments, the outer cross-sectional profile of the second dilator is circular, oval, or triangular in shape. In certain embodiments, the distal end 1523 of the second dilator 1508 incorporates a less steep inferior beveled surface 1509 than a first dilator 1500 bevel 1501 and a rounded tip 1510 to create an atraumatic tapered profile. In certain embodiments, the distal end of the second dilator minimizes tissue and nerve trauma during placement of dilation mechanisms. In certain embodiment, the distal end of second dilator 1508 incorporates a reference marking 1511 used to denote a side of second dilator 1508 that should face generally superior and tilted to match the angle of an exiting nerve root 0102. Certain embodiments of second dilator 1508 comprise an oblong hole 1512 spanning the length of the instrument to match the outer oblong cross-section of first dilator 1500. In certain embodiments, the proximal end 1524 of the second dilator 1508 comprises grooves 1513. In certain embodiments, the second dilator 1508 incorporates depth markers.

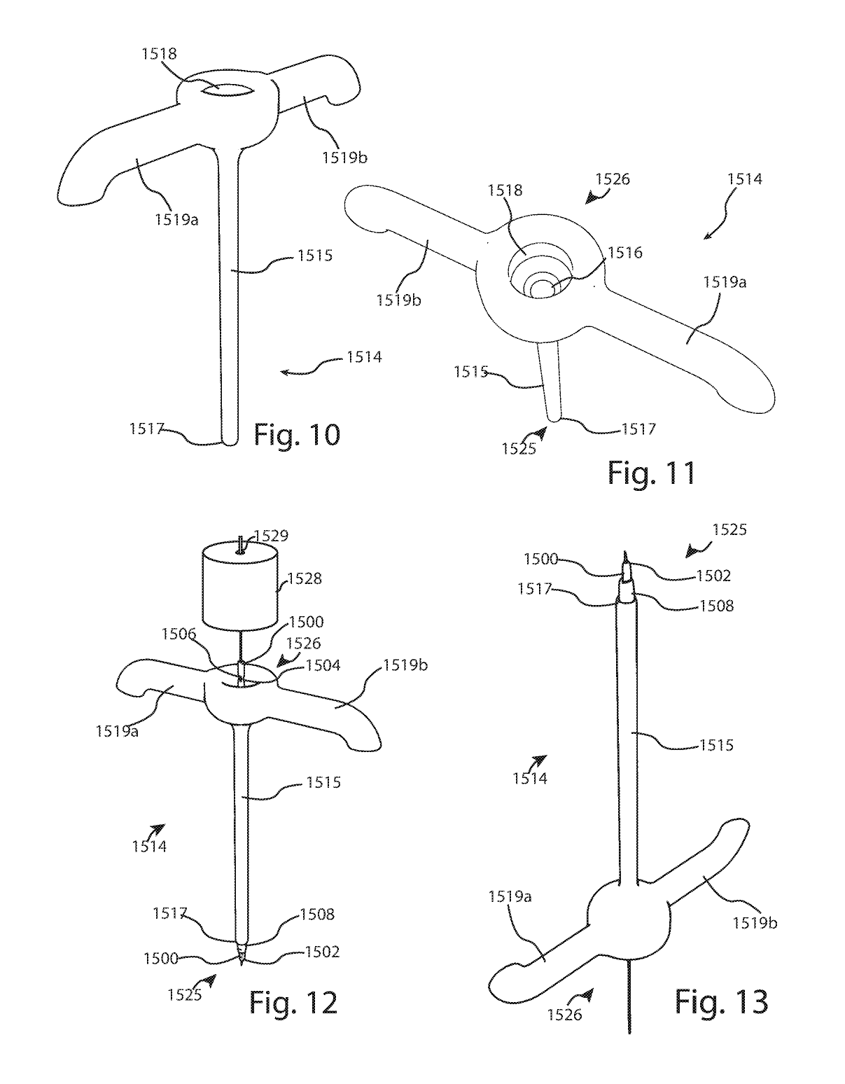

As seen in FIGS. 10-13, in certain embodiments, a sheath 1514 covers a first dilator, and one or more second dilators. In certain embodiments, the sheath shields the pathway to the target area to protect surrounding nerves. In certain embodiments, the sheath shields external structures from being physically affected by the passage of instrumentation and/or one or more expandable or non-expandable interbody cages through the pathway. In a certain embodiment, the sheath is an elongate tube. In certain embodiments, the material of the sheath includes stainless steel, titanium, aluminum, and other metals, and in certain embodiments, it will be appreciated that other materials, including but not limited to plastics and polymers are used. In certain embodiments, a sheath of any size is used. In certain embodiments, the sheath has an external diameter ranging between 12 mm and 8 mm. In certain embodiments, the sheath has an internal diameter ranging between 10 mm and 6 mm. In certain embodiments, a sheath has an external diameter no greater than 12 mm.

Referring to FIG. 12, in certain embodiments, the sheath 1514 is slidable and removable relative to the first dilator 1500 and/or the second dilator 1508. Referring to FIG. 11, FIG. 12, and FIG. 13, in certain embodiments, the sheath 1514 has a shaft 1515 and an oval shaped protrusion or a handle 1519a, 1519b. In certain embodiments, the length of the shaft 1515 is approximately 220 mm, with an outer diameter of 10.5 mm, although other sizes may also be used. Referring to FIG. 11 and FIG. 12, the shaft 1515 includes a cannula 1516 connecting a proximal end 1526 and a distal end 1525. In certain embodiments, the sheath cannula has a diameter of approximately 9 mm. In certain embodiments, the sheath 1514 distal end 1525 has a rounded tip 1517. The rounded tip 1517 minimizes tissue damage and nerve disruption while passing through Kambin's Triangle and other tissues. In certain embodiments, the sheath 1514 includes a hydrophobic coating. Referring to FIG. 11, in certain embodiments, the proximal end 1526 of a sheath 1514 incorporates a hole or opening 1518. In certain embodiments, a cannula 1516 is located between a distal end 1525 and proximal end 1526, where the cannula 1516 is connected with opening 1518. For example, the opening 1518 has a surface that tapers towards the cannula 1516. Certain embodiments of the sheath 1514 have a handle, such as a T-shaped handle, at the proximal end. In certain embodiment, the proximal handle incorporates an oval-shaped protrusion 1519a perpendicular to the axis of circular shaft 1515 and located around the large hole 1518. A second oval shaped protrusion 1519b is oriented 180 degrees from a first oval cross-sectioned protrusion 1519a with respect to the large hole 1518. In a certain embodiment, oval shaped protrusions 1519a and 1519b improve grip.

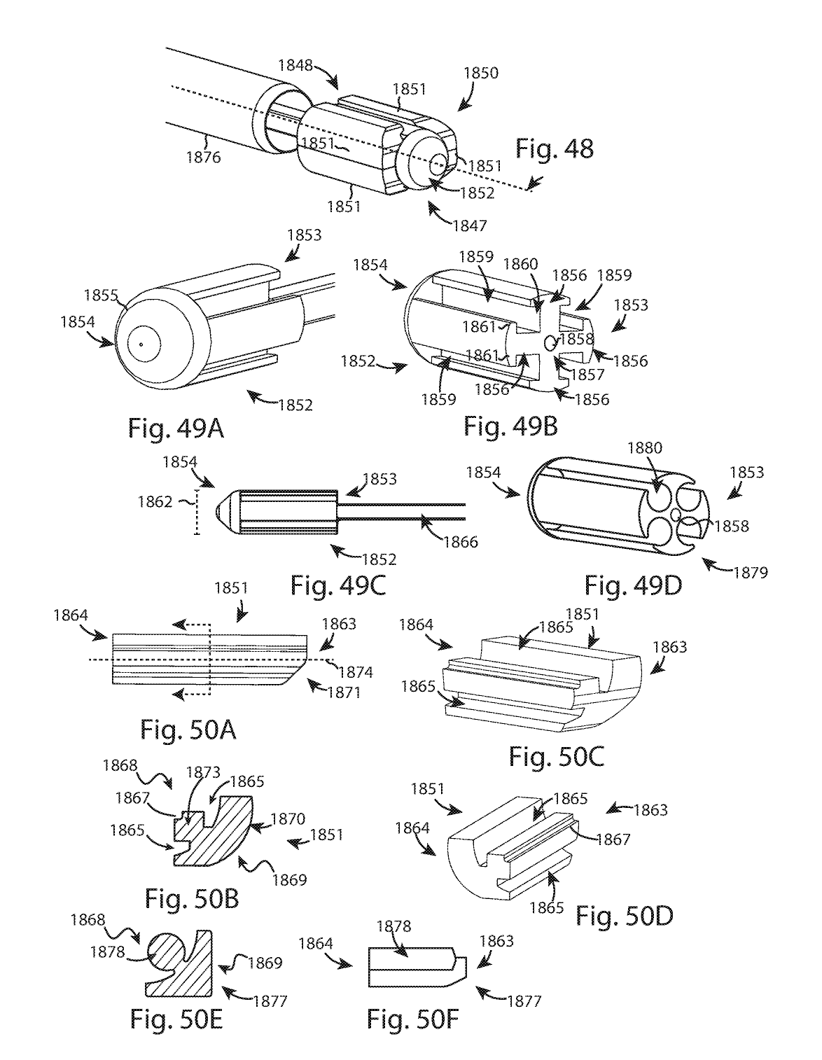

In certain embodiments, an implant includes an expandable interbody cage 1000 is placed into the space between vertebral discs. Referring to FIG. 14, in certain embodiments, the expandable interbody cage 1000 includes two long structural elements or center links 1100, and four short elements or end links 1200. In certain alternative embodiments, an expandable interbody cage 1750 includes four center links that separate from each other during deployment as shown in FIGS. 40A-F and FIGS. 41A-F. In certain embodiments, the arrangement of the structural elements allows a center link to contact a vertebral endplate when the expandable interbody cage 1000 is deployed. Referring to FIG. 14, in certain embodiments, a distal end 1226 end link 1200c is connected with a center link 1100 distal end, and a proximal end 1227 end link 1200d is connected with a center link 1100 proximal end. In certain embodiments, a center link and end link are hingeably connected. In certain embodiments, a first end link is hingeably connected with a second end link. In certain embodiments, pulling on a distal end towards the proximal end causes the center link to expand or extend in a direction away from a longitudinal axis 1228 of an implant or cage. In certain embodiments, a stem or an internal rod guides the proximal end 1227 end link 1200 and a distal end 1226 end link 1200. Referring to FIG. 14, in certain embodiments, the end links 1200 are arranged in pairs that form load-bearing hinges. In alternative embodiments, the system may incorporate one or more non-expandable interbody implants each comprising a singular solid structure. In certain embodiments, an implant comprises an assemblable interbody cage 1850 comprising two or more wedges 1851, as shown in FIG. 48. As used herein, the term “assemblable” means “able to be assembled during and/or following placement within an interbody space.” In certain embodiments, the material of the expandable interbody cage includes, but is not limited to titanium, polyetheretherketone (PEEK), carbon fiber, and/or stainless steel.

As seen in FIG. 15, certain embodiments of a center link 1100 have a lateral surface 1101, a ridged surface 1102, a hinge portion 1103, and a hole 1104. In certain embodiments, a ridged surface 1102 is shaped to engage one or more vertebral endplates. In certain embodiments, a ridged surface of a center link 1100 provides for increased purchase with one or more vertebral endplates. In certain embodiments, the purchase stabilizes an expandable interbody cage 1000 following deployment, preventing its within the interbody space.

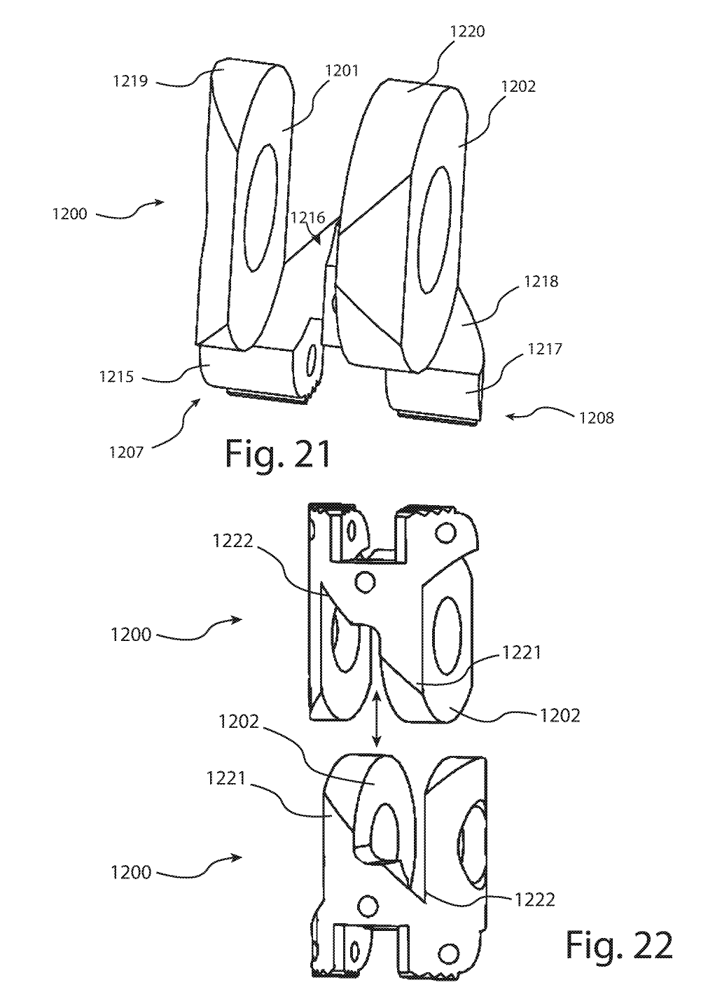

As seen in FIG. 16, in certain embodiments, center link 1100 includes a first radius cutout 1105, a second radius cutout 1106, and an interior surface 1107. As seen in FIG. 14, first radius cutout 1105 is shaped to mate with first convex surface 1215 and second convex surface 1217 of end link 1200, as depicted in FIG. 14. Referring to FIG. 16, second radius cutout 1106 is shaped to mate with curvature of external protrusion 1201 and internal protrusion 1202, for example, support surface 1219 of the external protrusion 1201 and support surface 1220 of internal protrusion 1202 as seen in FIG. 21. Referring to FIG. 14 and FIG. 16, a cutout 1108, also referred to as a groove, cuts into interior surface 1107 along its axial dimension allows slideable movement of an internal rod 1300 (seen in FIG. 14 and FIG. 24) in certain embodiments. In certain embodiments, an internal rod is referred to as a “stem.”

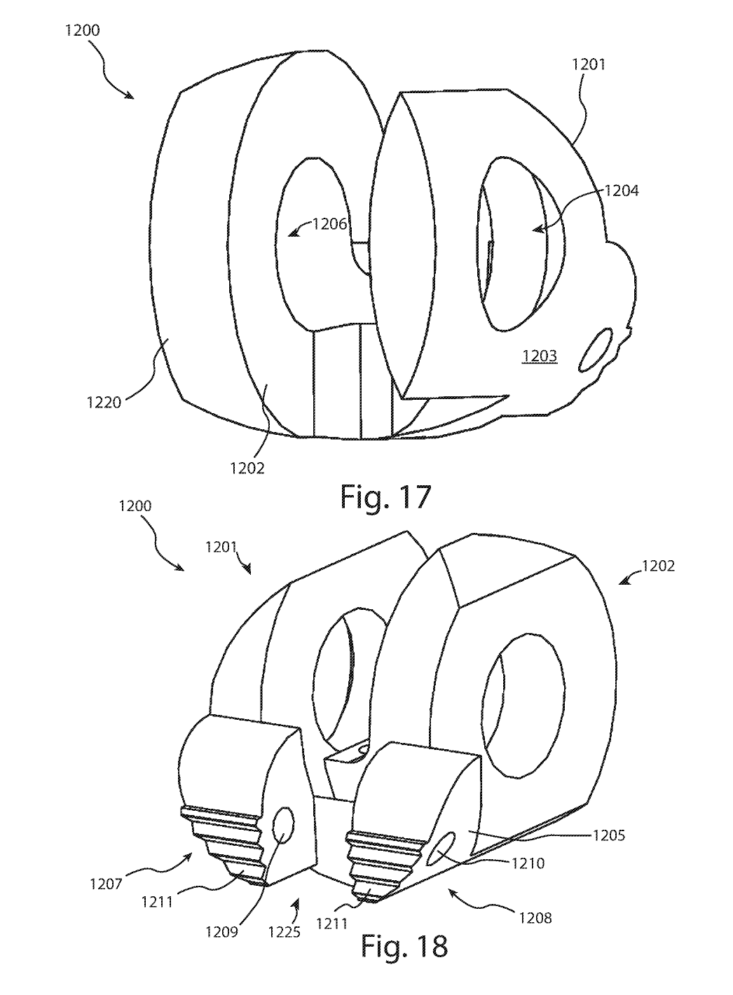

As seen in FIG. 17, in certain embodiments, an end link 1200 has an external protrusion 1201 and an internal protrusion 1202. External protrusion 1201 incorporates an outer short lateral surface 1203 and a dowel passage 1204. Internal protrusion 1202 has a dowel passage 1206. An internal protrusion has a support surface 1220 having a rounded shape to promote an axial rotation around a pin inserted in an inner hinge passage 1206 without obstruction, in certain embodiments.

As seen in FIG. 18, in certain embodiments, an end link 1200 has a first protrusion or first knuckle 1207, and a second protrusion or second knuckle 1208. A first knuckle 1207 has a pinhole 1209. Second knuckle has a 1208 has a lateral surface 1205 and pinhole 1210. In certain embodiments, a first knuckle 1207 and second knuckle 1208 have a ridged surface 1211. Still referring to FIG. 18, a gap 1225 is located between a first knuckle 1207 and second knuckle 1208.

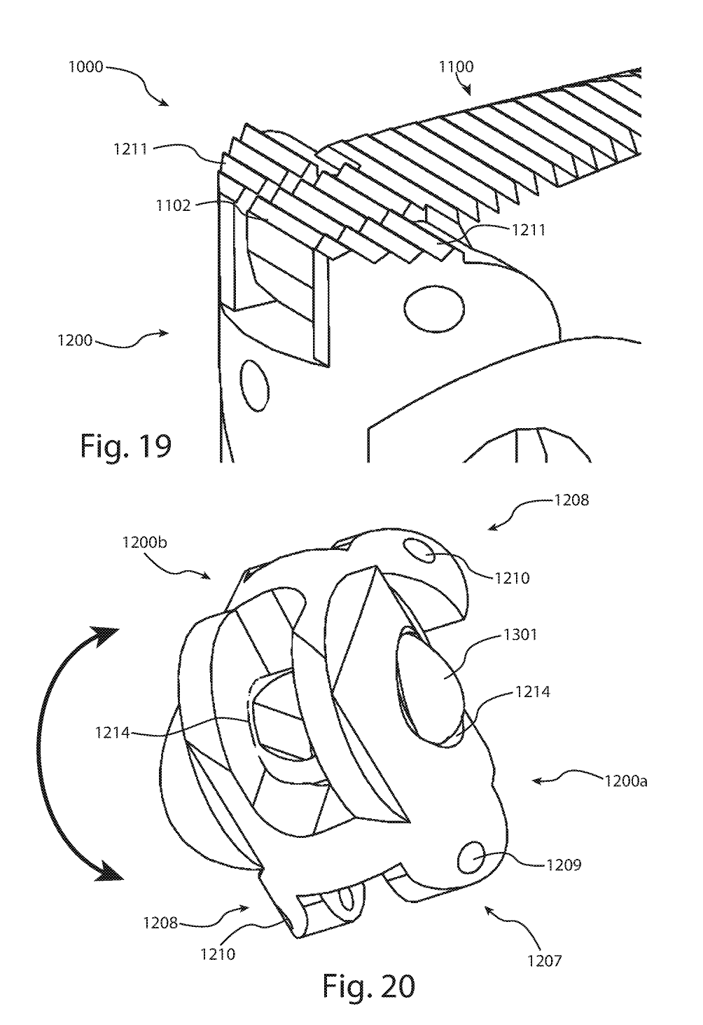

As seen in FIG. 19, in certain embodiments, an end link 1200 ridged surface 1211 is oriented obliquely to center link 1100 ridged surface 1102, such that rotation of end link 1200 when an expandable interbody cage 1000 is in a deployed or expanded configuration, ridged surface 1211 and long ridged surface 1102 form a generally contiguous surface. In certain embodiments, when an expandable interbody cage 1000 is in a deployed configuration, the end link 1200 ridged surface 1211 is substantially planar with a center link 1100 ridged surface 1102.

In certain embodiments, as seen in FIG. 20, each end link has a pinhole 1209 and pinhole 1210. As seen in FIG. 20, a first end link 1200a is paired with a second end link 1200b. In certain embodiments, a first end link and second end link are identical. In certain embodiments, one end link can be inverted and mated with another end link, where a dowel is placed through dowel openings 1214 of a first end link 1200a and second end link 1200b. In certain embodiments, a proximal dowel 1301 is placed through a first end link 1200a and a second end link 1200b. The first end link 1200a and the second end link 1200b are thus able to rotate around the dowel and relative to each other.

As depicted in FIG. 21, in certain embodiments, a first knuckle 1207 has a first convex surface 1215 and a first concave surface 1216, and a second knuckle 1208 has a second convex surface 1217 and second concave surface 1218. The outer surface of external protrusion 1201 has an external support surface 1219. Internal protrusion 1202 has an internal support surface 1220. The external support surface 1219 provides a load bearing surface area. In certain embodiments, the curvature of the first concave surface 1216, internal support surface 1220, second concave surface 1218, and external support surface 1219 are substantially the same, allowing the surfaces 1216, 1218, 1219, 1220 of one end link to rotate relative to a the surfaces 1216, 1218, 1219, 1220 of another end link. In certain embodiments, contacts between first concave surface 1216 on a first end link 1200 and internal support surface 1220 on a second end link 1200, and between second concave surface 1218 on a first end link 1200 and external large support surface 1219 on a second end link 1200 are load bearing. Thus, in certain embodiments, the present inventors have recognized that load is distributed among a first end link 1200a to a second end link 1200b when expandable interbody cage 1000 is in a deployed state.

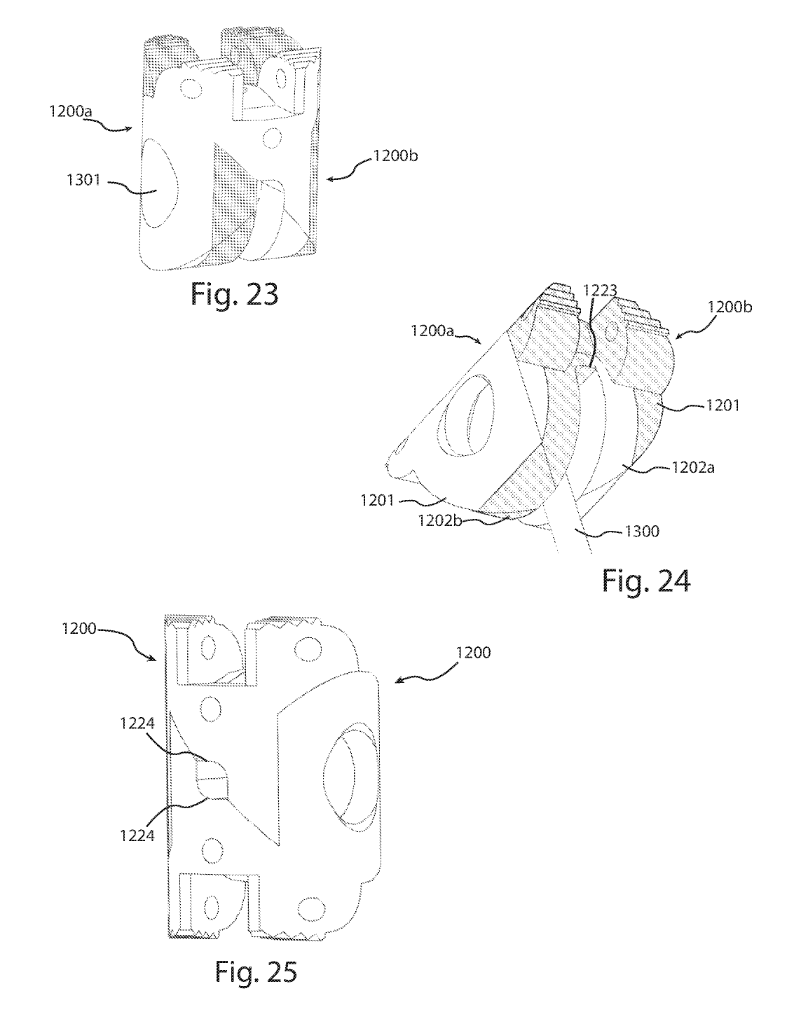

As depicted in FIG. 22, in a certain embodiment of the invention, the bowed exterior surface of internal protrusion 1202 meets the bowed exterior of end link 1200 at an angle, forming an angled projection 1221. A first end link has a wedge cut 1222 able to receive an angled projection 1221 of a second corresponding end link when mated, creating a tight fit between the first and second end link, as shown, for example, in FIG. 22 and FIG. 23, creating a tight fit between a first end link 1200a and second end link 1200b. In an embodiment, the position of angled projection 1221 and wedge cut 1222 halt rotation when a first end link 1200a and a second end link 1200b have rotated 180 degrees relative to each other. In alternative embodiments, the form factor of these elements may halt rotation at alternative positions, such as angles greater than 180 degrees.

As seen in FIG. 23, in certain embodiments, a first end link 1200a is shaped to mate with a second, inverted end link 1200b. When mated in the configuration seen in FIG. 23, both subunits are in a reference position, which is referred to as zero degrees of rotation relative to each other. From this position, both subunits are able to rotate around a dowel, such as a proximal dowel 1301 seen in FIG. 23. In an embodiment, both subunits are able to rotate to a final position of 180 degrees relative to each other.

As seen in FIG. 24, in certain embodiments, the space between a first internal protrusion 1202a on a first end link 1200a and a first external protrusion 1201a on a first end link 1200a is of the corresponding shape and dimensions to mate with a second internal protrusion 1202b from a second, inverted end link 1200b. The space between a first internal protrusion 1202a and a second internal protrusion 1202b is specifically dimensioned to accommodate an internal rod 1300. End link 1200 further incorporates transit shelf 1223. Transit shelf 1223 braces an end link 1200 against an internal rod 1300 when a first end link 1200a and a second end link 1200b are in transit position. In certain embodiments, internal rod 1300 spans the length of expandable interbody cage 1000.

As seen in FIG. 25, in certain embodiments, end link 1200 further incorporates a cutout or a deploy shelf 1224. Deploy shelf 1224 is a passage that is formed when a first end link 1200a and a second end link 1200b are mated in a deploy position. The form factor of a first end link 1200a and a second end link 1200b are such that a hole is formed when the two subunits are mated, allowing an internal rod 1300 to traverse. Curvature of a cutout or a deploy shelf 1224 is designed to accommodate internal rod 1300 while a first end link 1200a and a second end link 1200b are in a deployed state.

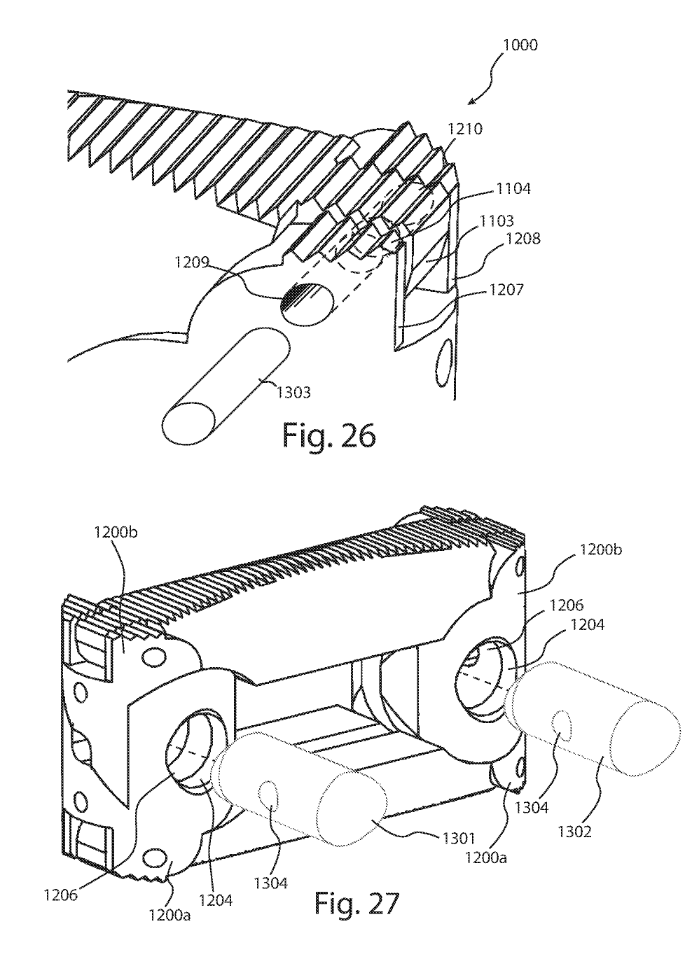

As seen in FIG. 26, in a certain embodiment, expandable interbody cage 1000 is assembled such that hinge portion 1103 is positioned between first protrusion 1207 and second protrusion 1208, which positions outer pinhole 1209, hole 1104, and inner pinhole 1210 in alignment and allows a pin 1303 to be inserted through the entire width of the expandable interbody cage 1000, forming a joint. This assembly allows end link 1200 and center link 1100 to rotate around pin 1303.

As seen in FIG. 27, in a certain embodiment, a channel is formed by outer dowel passage 1204 and inner dowel passage 1206 when a second end link 1200 is inverted and mated with a first end link 1200. The channel formed is of the appropriate dimensions to mate with a proximal dowel 1301 or a distal dowel 1302. Proximal dowel 1301 and distal dowel 1302 each act as the pin of a hinge, allowing a first end link 1200 and a second end link 1200 to rotate around a proximal dowel 1301 or a distal dowel 1302 relative to each other. Proximal dowel 1301 and distal dowel 1302 further incorporate dowel perforation 1304, which is of the corresponding dimensions to mate with internal rod 1300.

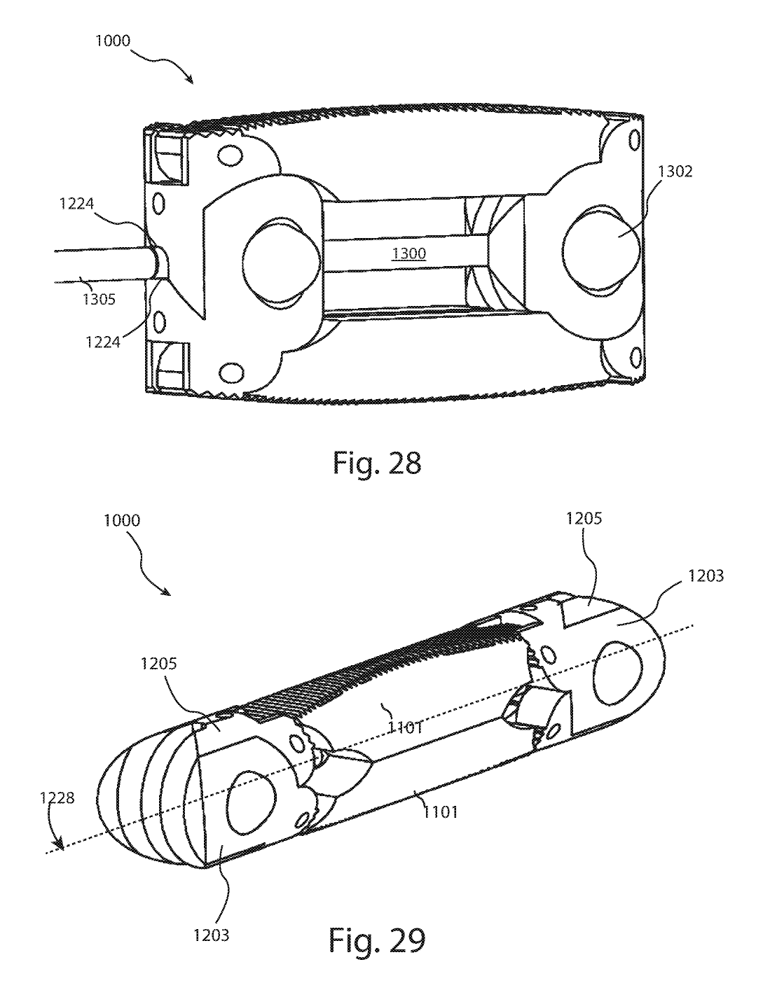

In certain embodiments, as seen in FIG. 28, internal rod 1300 is fixedly attached to distal dowel 1302. Internal rod 1300 spans the length of the expandable interbody cage 1000 and exits the proximal end through the channel formed between a first deploy shelf 1224a and a second deploy shelf 1224b when expandable interbody cage 1000 is in deployed configuration. At a position proximal to expandable interbody cage 1000, internal rod 1300 removably engages transit rod 1305. In the preferred embodiment, the removable engagement takes place via threaded surfaces.

When in transit form or retracted configuration, as seen in FIG. 29, varying embodiments of expandable interbody cage 1000 have a rounded profile when viewed from the axial dimension that is able to pass through a sheath 1514 or cannula of the corresponding dimensions. In certain embodiments, an expandable interbody cage 1000 includes an elongated form extending from a proximal end to a distal end. In certain embodiments, components of expandable interbody cage 1000 are sequentially stacked within the sheath 1514 prior to placement within the interbody space, as depicted in FIGS. 48-53. In certain embodiments, sequentially stacked components incorporate directionally tapered ends forming wedges that controllably slide against each other into different intended areas of the interbody space. In certain embodiments, a rounded profile is formed from long lateral surface 1101, outer short lateral surface 1203 and inner short lateral surface 1205. In certain embodiments, the rounded profile makes efficient use of structural material in the expandable interbody cage 1000 that enables fit through a narrow, rounded passage. In certain embodiments, the rounded profile also increases radial adjustability around the axis of the expandable interbody cage 1000. In certain embodiments, the diameter of the rounded profile is 9 millimeters, enabling the expandable interbody cage 1000 to fit into a sheath 1514 having an inner diameter of approximately 9 mm. It will be appreciated that in varying embodiments, a diameter of the expandable interbody cage 1000 in transit mode or configuration is between 7 mm and 12 mm. In alternative embodiments, the axial profile of the expandable interbody cage 1000, and correspondingly the sheath 1514, is substantially oval, substantially rectangular, or substantially rectangular with rounded edges in shape, corresponding to the parameters of the generally oblong boundary of Kambin's Triangle.

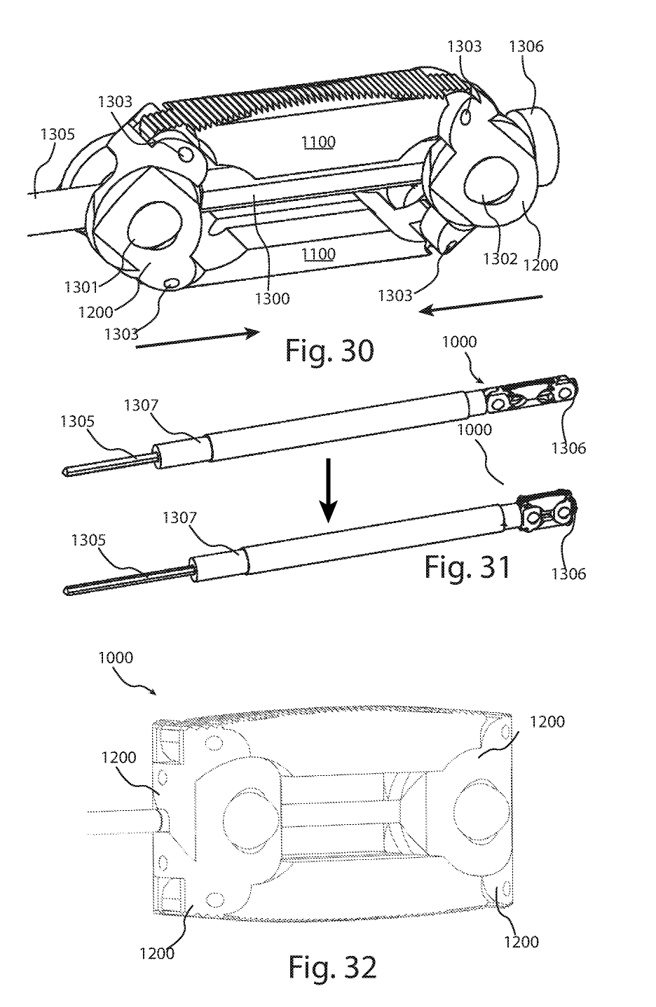

In varying embodiments, expandable interbody cage 1000 is transformable from a transit mode into a deployed mode. As seen in FIG. 30, in certain embodiments, end links 1200 rotate around a proximal dowel 1301 or distal dowel 1302 during a shift between transit mode and deployed mode. End links 1200 slide along internal rod 1300 towards the center, decreasing overall length of expandable interbody cage 1000 and increasing the distance between center links 1100. Compression of the expandable interbody cage 1000 in its axial direction translates to a force in a vertical dimension through the rotatable joints. This force in the vertical direction drives center links 1100 away from each other. Transit rod 1305 is removably engaged with internal rod 1300, such as by threads. Internal rod 1300 is further engaged with distal plate 1306. In certain embodiments, as described and shown for FIGS. 40A-F and FIGS. 41A-F, an implant includes an expandable interbody cage 1750 that transforms from a transit or retracted configuration to a deployed or expanded configuration.

In certain embodiments, portions and features of an implant are able to rotate to transition between a transit configuration and a deployed configuration. In certain embodiments, an implant as described in the following references are used during the methods associated with a deliver apparatus step 1405, and deploying a cage step 1406: U.S. Pat. No. 8,034,109 to Zwirkoski and filed Feb. 24, 2006, U.S Patent Publication No. 2006/0265077 to Zwirkoski and published Nov. 23, 2006, and U.S. Patent Publication No. 2012/0016481 to Zwirkoski and published 2012 Jan. 19, all of which are incorporated herein by reference. It will be appreciated that in certain embodiments, portions or features of an implant or cage are rotated in order to deploy the implant or cage.

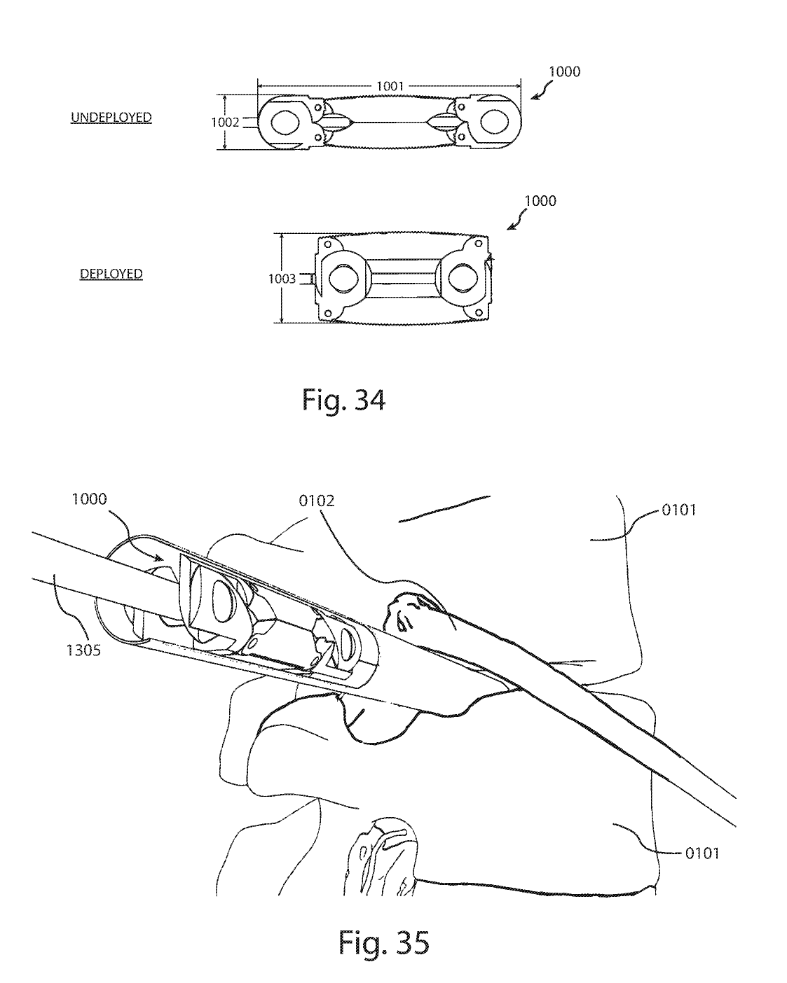

As seen in FIG. 34, in a certain embodiment, expandable interbody cage 1000 comprises transit length 1001 and transit height 1002 when in transit mode, and deploy height 1003 when in deployed mode. Dimensions of center links 1100 and links 1200 may vary as required for different distraction heights. In a certain embodiment, expandable interbody cage 1000 comprises a transit length 1001 of 35 millimeters, transit height of 9 millimeters, and a deploy height 1003 of 12 millimeters in deployed configuration. In alternative embodiments, the transit form may comprise 35 millimeters transit length 1001 and 13 millimeters deploy height 1003 in deployed form; 37 millimeters in transit length 1001 and 14 millimeters in deployed height 1003; or 37 millimeters in transit length 1001 and 15 millimeters in deployed height 1003. These dimensions are not comprehensive of all possible embodiments, and are strictly meant to serve as example embodiments for clarity.

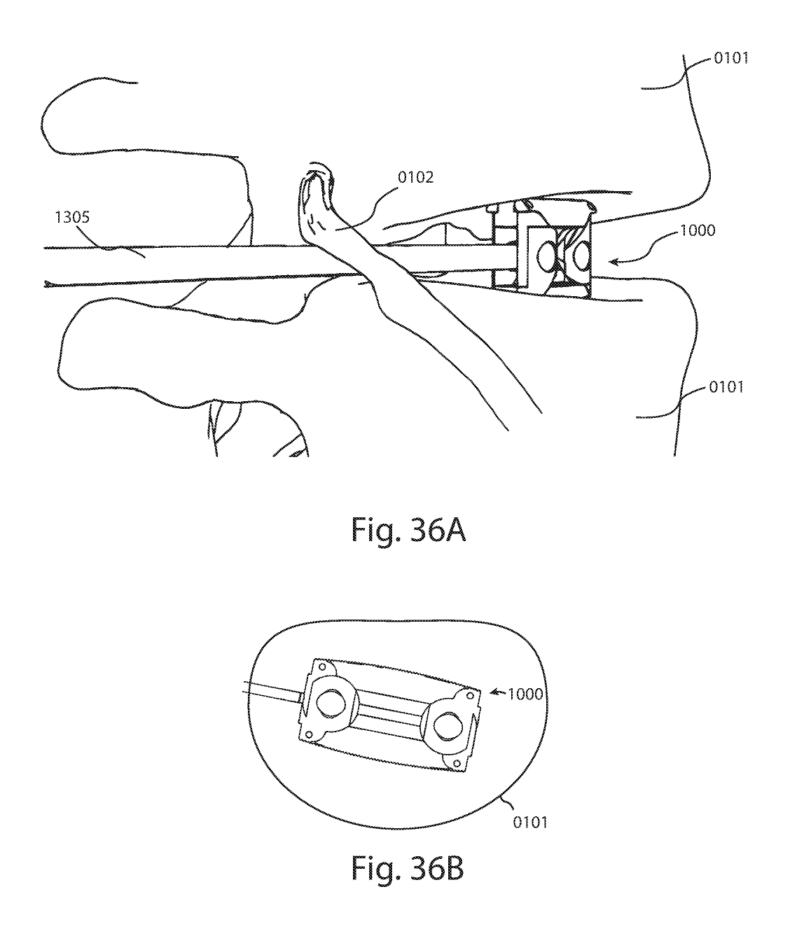

As seen in FIG. 35, in certain embodiments, expandable interbody cage 1000 in transit form is protected from neural and other soft tissue. Transit rod 1305 is used to advance expandable interbody cage 1000 over K-wire, through the sheath 1514, and into an interbody space. In a certain embodiment, expandable interbody cage 1000 is safely advanced through a sheath 1514 placed between the structures comprising Kambin's Triangle in this way, without nerve impaction. As seen in FIG. 36, in a certain embodiment, expandable interbody cage 1000 positioned in an interbody space, once safely through Kambin's Triangle and deployed, distracts two vertebral bodies 0101. Following distraction, transit rod 1305 is safely removable through the sheath 1514.

As seen in FIG. 31, in certain embodiments, the system incorporates a deployment tool or instrument. In a certain embodiment, the inserter operates to deploy an expandable interbody cage 1000 by mechanically transforming said expandable interbody cage 1000 from an undeployed (or retracted configuration) to a deployed (or expanded) configuration. The inserter attaches to an expandable interbody cage 1000 in certain embodiments through a threaded end designed to threadably engage with the expandable interbody cage 1000 to hold it. In certain embodiments, the inserter is a deployment tool that incorporates or abuts a tubular protrusion 1307 to facilitate the transfer of force. In a certain embodiment, the deployment tool incorporates a substantially tubular protrusion of the appropriate dimensions to fit through a low-diameter sheathed passage. In a certain embodiment, the deployment tool consists of a substantially elongate shape of a diameter to fit through the sheath 1514. In certain embodiments, the deployment tool applies force to transit rod 1305. In the preferred embodiment, the deployment tool functions to apply force through a mechanism substantially similar to a pop rivet gun. In certain embodiments, force on a transit rod 1305 is translated to distal plate 1306, and a compression force is generated between distal plate 1306 and tubular protrusion 1307. In certain embodiments, within the expandable interbody cage 1000, said compression force is translated through rotatable joints, and forces a change in configuration of the implant from transit configuration to deploy configuration. In certain embodiments, compressive force applies to expandable interbody cage 1000 as tubular protrusion 1307 pushes on proximal end links 1200, while transit rod 1305 pulls on distal plate 1306.

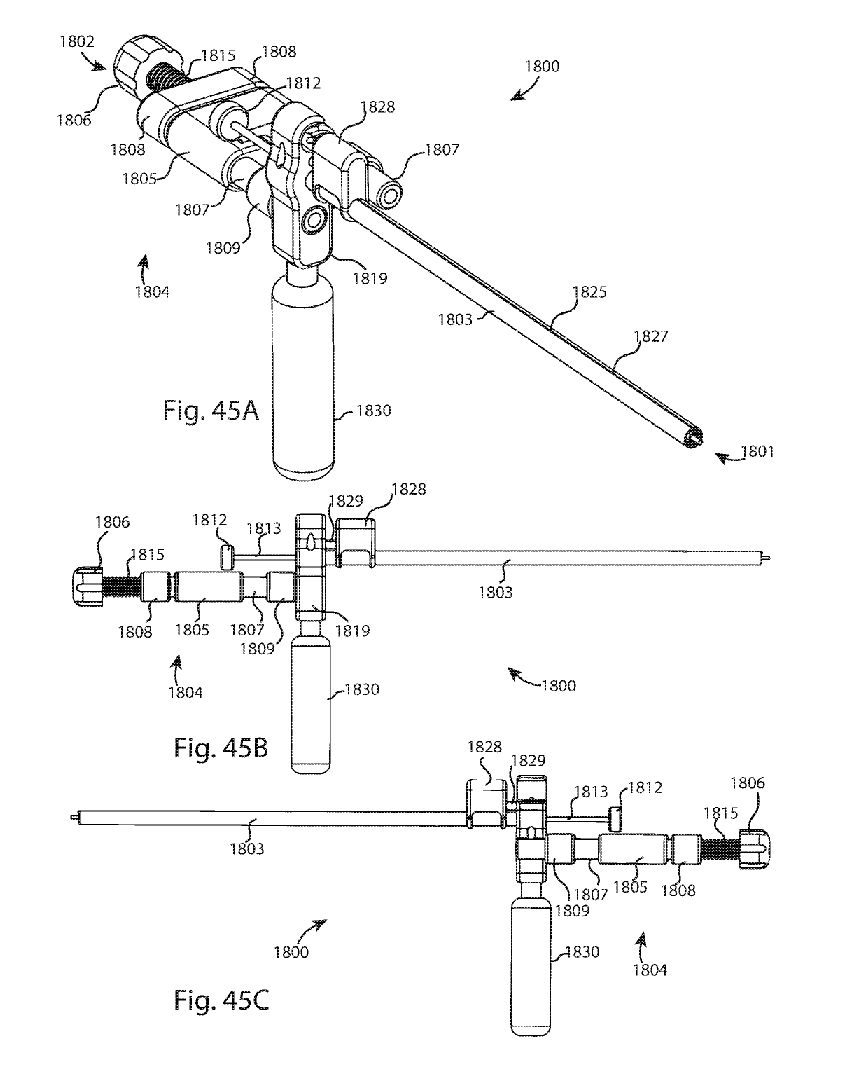

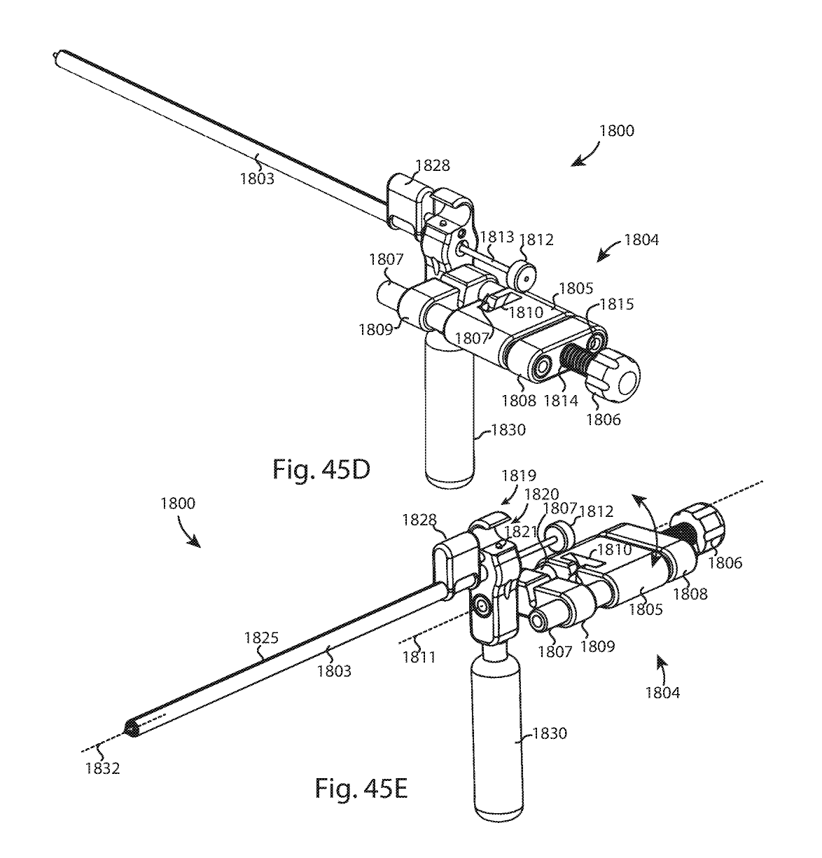

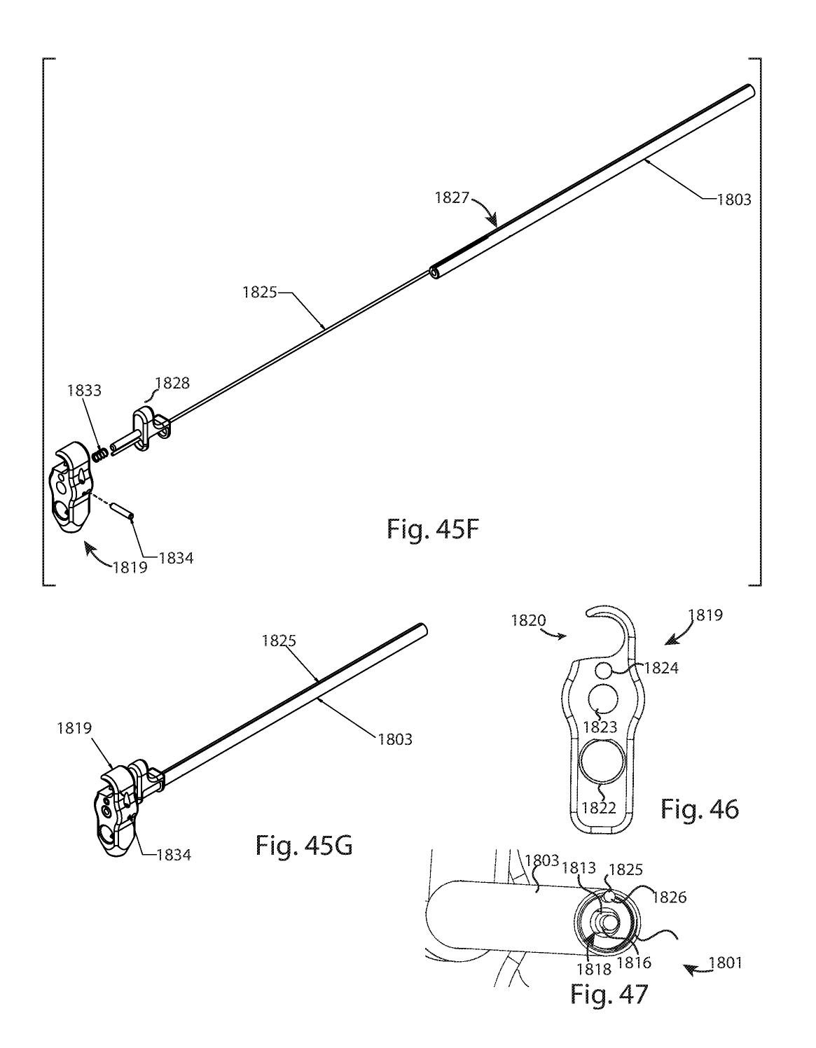

In certain embodiments, an inserter, such as a deployment tool 1800 shown in FIGS. 45A-G allows delivery of implant. In certain embodiments, a deployment tool 1800 includes a distal end 1801 and a proximal end 1802. A delivery sheath 1803 located towards the distal end 1801 allows placement of an implant or cage in the surgical site. In certain embodiments, a proximal end 1802 includes a delivery assembly 1804. In certain embodiments, a delivery assembly 1804 includes a retention block 1805 threadably attached to an adjustment bolt 1806. In certain embodiments, a guide column 1807 is disposed between a first block 1808 and second block 1809, where a retention block 1805 is slideably connected with the guide column 1807. Referring to FIG. 45D, in certain embodiments, a first block 1808 has a threaded opening 1814 that is threadably engaged with threads 1815 of an adjustment bolt 1806. In certain embodiments, an adjustment bolt 1806 is further rotatably connected with the retention block 1805. Rotation of the adjustment bolt 1806 allows slideable adjustment of the retention block 1805 along a guide column 1807. The guide column 1807 is oriented in a direction that is generally parallel with an axis 1811, which runs in a generally longitudinal direction.

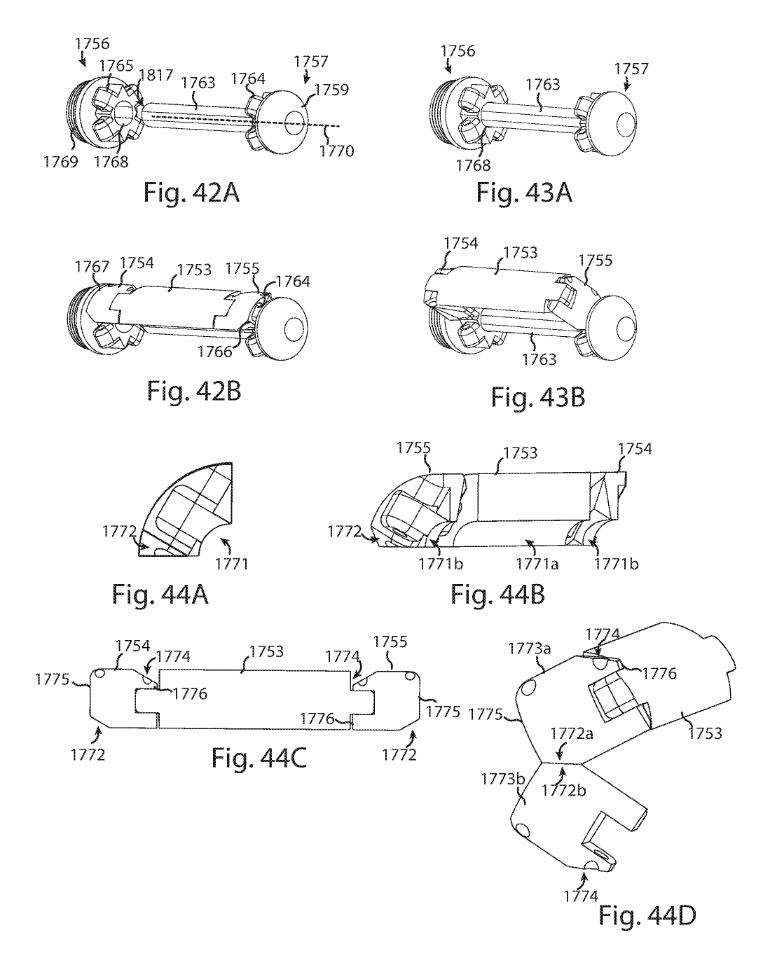

Referring to FIGS. 45D-E, in certain embodiments, a retention block 1805 includes a retention hole 1810 that retains a portion of the deployment tool 1800. In certain embodiments, a retention hole 1810 accommodates for example, a stem knob 1812. In certain embodiments, a stem knob 1812 is connected to a stem connector 1813. The stem connector 1813 is passed through a delivery sheath 1803 and has an end located near a deployment tool distal end 1801, for example, near a distal end of a delivery sheath 1803. Referring to FIG. 47, in certain embodiments, the stem connector 1813 end 1818 includes a tip 1816 that threadably engages with a corresponding threaded opening located on an expandable interbody cage. In certain embodiments, a corresponding threaded opening includes opening 1817 shown in FIG. 40C, where the opening 1817 is located on the stem 1763 as seen in FIG. 42A. In certain embodiments, the threaded tip 1816 engages with distal plate 1306 as shown in FIG. 30. In certain embodiments, threaded tip 1816 engages with a dowel perforation 1304 as shown in FIG. 27, where a dowel perforation 1304 includes a threading. In certain embodiments, attachment of the stem connector 1813 tip 1816 to an expandable interbody cage is through a slot and hole connection.

In certain embodiments, a base tool block 1819 is connected to a delivery assembly 1804. In certain embodiments, base tool block 1819 is further connected with a delivery sheath 1803. In certain embodiments, a delivery assembly 1804 pivots about an axis 1811, which is, for example, located about a longitudinal axis of a guide column 1807. A base tool block 1819, in certain embodiments, includes a retention element 1820 that captures a portion of the delivery assembly 1804. In certain embodiments, as shown in FIG. 45E, the retention element 1820 retains the guide column 1807 when the instrument is in a closed position. In certain embodiments, a spring-actuated pin 1821 located within or near a retention element 1820 further restricts movement of delivery assembly 1804 when the instrument is in a closed position. In a closed position, the delivery assembly 1804 restricts slideable movement of a stem knob 1812 and stem connector 1813, until the stem knob 1812 and stem connector 1813 are further adjusted by moving the retention block 1805. In certain embodiments, rotation of the adjustment bolt 1806 controls the location of the retention block 1805, which retains the stem knob 1812, thus controlling the location of the stem connector 1813 end 1818.

In certain embodiments, referring to FIGS. 45E and 46, a base tool block 1819 is pivotably connected with a delivery assembly 1804. In certain embodiments, a portion of a guide column 1807 is placed through a first opening 1822 of a base tool block 1819. In certain embodiments, a stem connector 1813 is passed through a second opening 1823 of a base tool block 1819. In certain embodiments, a delivery sheath 1803 is joined with the second opening 1823 of the body 1819.

In certain embodiments, a locking pin 1825 is laid along the delivery sheath 1803. Referring to FIG. 47, the tip 1826 of the locking pin 1825 is located at the distal end 1801 of the deployment tool 1800. In certain embodiments, a delivery sheath 1803 has a slit 1827 oriented in its longitudinal direction that accommodates the locking pin 1825. A locking pin 1825 is connected with a locking pin lever 1828. In certain embodiments, a locking pin lever 1828 is further guided into the base tool block 1819 with a guiding pin. For example, as shown in FIGS. 45B-C and 46, a connector 1829 is attached to the locking pin lever 1828, where the connector 1829 passes through a base tool block 1819 third opening 1824. In certain embodiments, a locking pin 1825 and/or a locking pin lever 1828 has a spring-actuated connection with, for example a base tool block 1819, as seen in FIG. 45F. Referring to FIG. 45F-G, a spring 1833 is placed between a locking pin lever 1828 and a base tool block 1819. A delivery sheath 1803 is attached to the locking pin lever 1828, and the delivery sheath is further secured to the base tool block 1819 with a fastener 1834. In certain embodiments, the locking pin 1825 engages with a pin cutout 1831 as shown, for example, in FIGS. 40A and 40C. Engagement of the locking pin 1825 with a pin cutout 1831 allows rotation of the implant or cage around the longitudinal axis 1832 of the delivery sheath 1803. Pulling the locking pin towards the proximal end of the delivery tool releases the locking pin engagement with the pin cutout 1831 of an implant or cage. In certain embodiments, a deployment tool 1800 has a handle 1830 to allow a user to hold the delivery tool. In certain embodiments, a handle 1830 is attached to a base tool block 1819. In certain embodiments, as shown in FIG. 47, the distal end interior surface of a delivery sheath 1803 has a thread 1835. In certain embodiments, delivery sheath 1803 thread 1835 allows attachment to an expandable interbody cage. In certain embodiments, the thread 1835 threadably engages with thread 1769 located on a proximal element 1756 of an expandable interbody cage, as seen in FIG. 42A. In certain embodiments, rotation of the deployment tool about the longitudinal axis 1832 allows release of the implant from the deployment tool. In certain embodiments, the deployment tool delivery sheath 1803 is rotatable about the longitudinal axis 1832, as shown in FIG. 45E.

As seen in FIG. 32, in certain embodiments, expandable interbody cage 1000, when in deployed configuration, provides structural support through end links 1200. In certain embodiments, expandable interbody cage 1000 can be used to distract two vertebral bodies during transformation from a transit configuration to a deployed configuration after insertion into an interbody space, as depicted by FIGS. 35 and 36A. In varying embodiments, ridges 1211, as shown for example in FIGS. 18-19 engage and create purchase with the surface of a vertebral end plate. In alternative embodiments, expandable interbody cage 1000 is oriented 90 degrees axially, such that the expansion of the expandable interbody cage 1000 occurs in a plane substantially parallel to the plane of the interbody space, as depicted by FIG. 36B.

In certain embodiments, an implant such as an expandable interbody cage 1750 shown in FIGS. 40A-F and FIGS. 41A-F is used. Referring to FIG. 40A and FIG. 41A, in certain embodiments, an expandable interbody cage 1750 has a proximal end 1751 and a distal end 1752. Referring to FIGS. 40D-E and FIGS. 41D-E, a plurality of links, including a center link 1753, and a proximal end link and a distal end link are disposed between a proximal end 1751 and a distal end 1752. In certain embodiments, pulling on a distal end towards the proximal end causes the center link to expand or extend in a direction away from a longitudinal axis 1770 (seen in FIG. 42A) of an implant or cage. In certain embodiments, a stem or an internal rod guides the proximal end 1751 end link 1754 and a distal end 1752 end link 1755. In certain embodiments, the center link 1753, the proximal link 1754, and distal link 1755 are disposed between a proximal element 1756 and a distal element 1757. When the expandable interbody cage 1750 is in an expanded configuration as shown in FIG. 41A-F, the center link 1753 assumes a position that increases the effective volume that the expandable interbody cage occupies. In a retracted state as shown in FIG. 40A-F, the outer diameter 1758 (shown in FIG. 40D) of the cage 1750 is sized to pass through a dilator of 9 mm, although it will be appreciated that the outer diameter 1758 can range from 3 mm to 15 mm in certain embodiments, and is of any size in certain embodiments. In certain embodiments, as shown in FIGS. 40A-F, the expandable interbody cage in a retracted configuration is generally cylindrical in shape. In certain embodiments, the expanded or deployed configuration has a substantially square or rectangular shape. In certain embodiments, the expanded or deployed configuration has a width that is generally greater than its height. Referring to FIG. 40F, the outer surface 1760, 1761, and 1762 of the center link 1753, proximal link 1754, and distal link 1755 have curved surface, although other types of surfaces can be used in other embodiments.

Referring to FIGS. 40D-E and FIGS. 41D, in certain embodiments, the distal element 1757 has a tip 1759. In certain embodiments, the tip 1759 includes a feature that allows a gradual, atraumatic opening of tissue, including, but not limited to, for example, a frustoconical shape, a bullet-nose shape, and a tapered shape. Referring to FIG. 42A, in certain embodiments, the distal element 1757 includes a tip 1759, a first hinge element 1764, and a stem 1763. Referring to FIG. 42B, the distal element 1757 first hinge element 1764 is hingeably connected to a distal link 1755 at a second hinge element 1766. In certain embodiments, a hinge element 1764 and a hinge element 1766 include knuckles, which are retained by a pin. Referring to FIG. 42A, in certain embodiments, a proximal element 1756 includes a hinge element 1765. Referring to FIG. 42B, the proximal element 1756 first hinge element 1765 is hingeably connected to a proximal link 1754 at a second hinge element 1767.

Referring to FIG. 42A, in certain embodiments, a proximal element 1756 includes thread 1769 and an opening 1768. In certain embodiments, a stem 1763 of the distal element 1757 passes through the opening 1768 of proximal element 1756. In certain embodiments, referring to FIGS. 41F and 43A, the stem 1763 passes through opening 1768 when the expandable interbody cage is in an expanded configuration. In certain embodiments, a cross-sectional profile of a stem 1763 keys in with the opening 1768 having a similar cross-sectional profile, preventing rotation of the distal element 1757 about a longitudinal axis 1770.

In certain embodiments, a distal link 1755 and center link 1753 are hingeably connected, for example, as shown in FIG. 43B. Still referring to FIG. 43B, in certain embodiments, a center link 1753 and a proximal link 1754 are hingeably connected. When in an expanded configuration, the distance between distal element 1757 and proximal element 1756 is decreased, which displaces the center link 1753 away from the stem 1763. In certain embodiments, as shown in FIGS. 41A-F, an expandable interbody cage 1750 includes a plurality of center links, distal links, and proximal links.

Referring to FIGS. 44A and 44B, in certain embodiments, the links have a cutout 1771, 1771a, b. It will be appreciated that a cutout has a shape to accommodate an internal rod or stem 1763, guide wire, or other objects. In certain embodiments, the cutout is radial. In certain embodiments, as seen in FIGS. 44A-D, a proximal link and/or distal link includes a notch 1772. In certain embodiments, when an expandable interbody cage 1750 is in an expanded configuration, a notch 1772a surface of a first link 1773a meets with a notch 1772b surface of a second link 1773b as seen in FIG. 44D. In certain embodiments, a notch 1772 is located on a first end 1775 of a proximal link or distal link, where the first end 1775 is connected with a distal element 1757 or proximal element 1756. In certain embodiments, a second end 1776 of a proximal link or distal link is connected with a center link. In certain embodiments, a second end 1776 includes a second notch 1774 as seen in FIG. 44C-D. In certain embodiments, a second notch 1774 surface meets with an upper or lower end plate when an expandable interbody cage 1750 is placed inside a disc space.

In certain embodiments, a trialing instrument includes a form as in an expandable interbody cage 1750 shown in FIGS. 40A-F and FIGS. 41A-F. In certain embodiments, a trialing instrument with a similar mechanism as described for FIGS. 40A-F and FIGS. 41A-F allows a trial implant to be placed in the vertebral disc space as to determine the correct size implant. A trialing instrument is inserted into the disc space, and expanded or deployed to determine whether the particular size is appropriate. The trialing instrument can further be retracted or collapsed and removed.

In certain embodiments, an implant includes an assemblable interbody cage 1850 comprising two or more wedges 1851, as shown in FIG. 48. In certain embodiments, two or more wedges 1851 are placed into position by being guided by a central component 1852. Referring to FIG. 48, assemblable interbody cage 1850 includes a form following a longitudinal axis 1849. In certain embodiments, an assemblable interbody cage 1850 includes a distal end 1847 and a proximal end 1848. Referring to FIG. 49A-C, a central component 1852 has a proximal end 1853 and a distal end 1854. A distal end 1854 has a tip 1855, where in certain embodiments, a tip includes a feature for a gradual, atraumatic opening of tissue. In certain embodiments, the feature includes, but is not limited to, for example, a frustoconical shape, a bullet-nose shape, and a taper. In certain embodiments, a central component 1852 includes a plurality of rails 1856. In certain embodiments, rails 1856 are positioned in a radially outward direction from the central component central stem 1857. A slot 1859 is formed in a space between the rails 1856. In certain embodiments, a slot 1859 has an opening 1860 connected with a proximal end of the central component. A rail 1856 further includes a retaining ledge 1861 in certain embodiments. In certain embodiments, a central component 1852 has a diameter 1862 that is adapted for use in an OLLIF approach. In certain embodiments, the diameter 1862 is approximately 9 mm, although it will be appreciated that the outer diameter can ranges from 3 mm to 15 mm in certain embodiments, and is of any size in certain embodiments. A stem 1866 attached to the central component central stem 1857. In certain embodiments, a central component includes an attachment hole 1858 located on a central component proximal end 1853, where a stem 1866 can attach to the central component. In certain embodiments, attachment of a central component to a stem is through a threaded connection.

Certain embodiments of the invention include two or more wedges 1851. Referring to FIG. 50A-D, a wedge 1851 has a proximal end 1864 and a distal end 1863 and oriented along a generally longitudinal axis 1874. In certain embodiments, a distal end 1863 has a ramped surface 1871, where a ramped surface helps to position a wedge into the disc space. A wedge 1851 has a rail cutout 1865 that accommodates an outer shape of a rail 1856. A wedge 1851 further includes a keyed element 1873 on the interior portion 1868, where the keyed element 1873 runs substantially along a longitudinal axis 1874. The keyed element 1873 further includes a stem cutout 1867 in certain embodiments. Referring to FIG. 50E-F, in certain embodiments, a wedge 1877 has a distal end 1863, a proximal end 1864, an interior portion 1868, and an exterior portion 1869. It will be appreciated that in certain embodiments, the exterior portion of a wedge is available in a number of different shapes, included having a rounded surface or a planar surface. In certain embodiments, a wedge 1877 has a keyed element 1878 that is rounded. It is contemplated that in certain embodiments, a keyed element 1878 of a wedge 1877 fits through a slot 1880 of a central component 1879 shown in FIG. 49D.

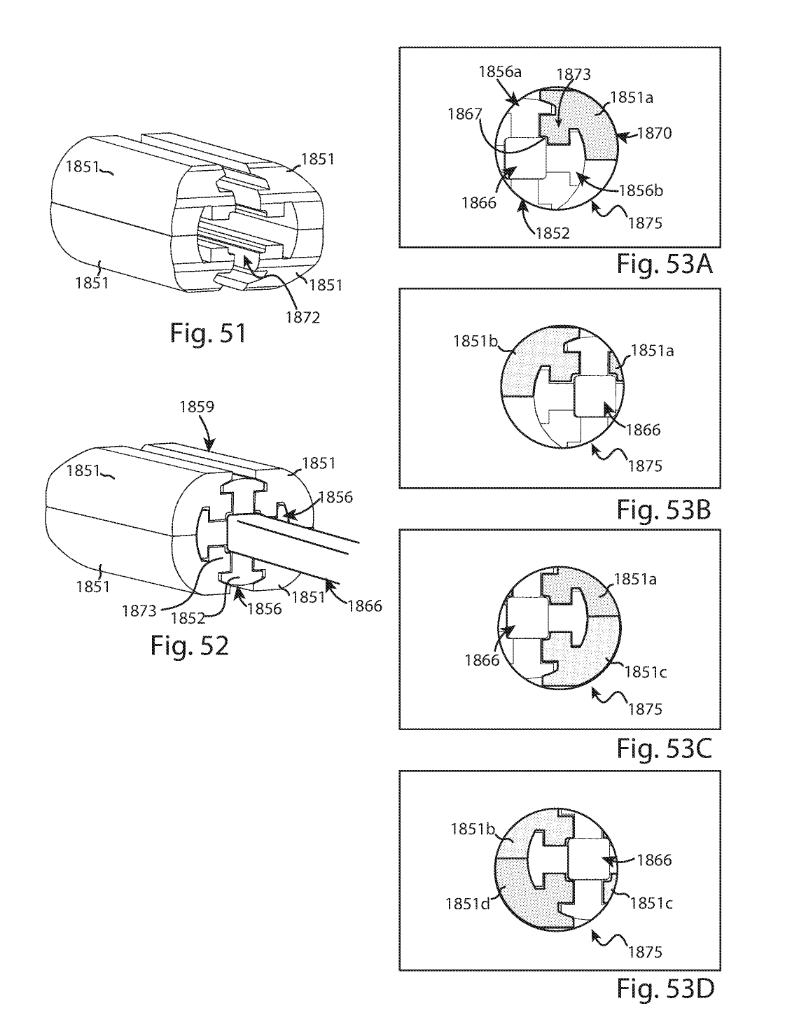

Referring to FIG. 51 showing a distal end perspective view of a plurality of wedges 1851, when properly assembled, a cavity 1872 is created among the wedge 1851 pieces. Referring to FIG. 52, a plurality of wedges 1851 are placed around a central component 1852, such that a central component 1852 is disposed in a cavity 1872 shown in FIG. 51. In certain embodiments, the keyed element 1873 of a wedge 1851 is placed within a slot 1859 of the central component 1852. Referring to FIGS. 49B and 52, the retaining ledge 1861 of the rail 1856 constricts the keyed element 1873 of a wedge 1851 to a movement that is generally along a longitudinal axis.

In certain embodiments, wedges 1851 are sequentially delivered to a vertebral disc space. Referring to FIGS. 53A-D, the wedges are placed through a working sheath. An exemplary view through a working sheath boundary 1875, where the implant is viewed from the proximal side, is shown in FIG. 53A-D. Referring to FIG. 53A, a first wedge 1851a is placed through the sheath boundary 1875, and positioned so that the keyed element 1873 fits between a first rail 1856a and a second rail 1856b. Referring to FIGS. 50B and 53A, a wedge has a surface profile 1870 located on an exterior portion 1869. Referring to FIG. 53A, the surface profile 1870 has a form matching that of a working sheath boundary 1875. Furthermore, still referring to FIG. 53A, the stem 1866 has an edge that engages with a stem cutout 1867 located on the wedge 1851a. Initially, the central component, which is attached to a stem, is passed through a working sheath 1876. Once the central component is in position, wedges are sequentially placed through the working sheath. As the wedge 1851a is passed through a working sheath 1876, the stem cutout 1867 and the surface profile 1870 help to guide the wedge 1851a along the stem and the working sheath. The wedge is pushed out of the working sheath, until the wedge reaches the appropriate quadrant of a central component 1852. The wedge is further pushed until it is engaged with the central component. Referring to FIGS. 53A-D, once a first wedge 1851a is positioned into a central component 1852, the sheath is repositioned in order to insert the other wedges 1851b, c, d.

In certain embodiments, the stem 1866 has a non-circular profile. In certain embodiments, a stem 1866 has a square cross section. In certain embodiments, the stem 1866 generally has a non-circular profile to allow guidance of a wedge through the working sheath. In certain embodiments, a stem includes a cross section with other shapes. It will be appreciated that in certain embodiments, a central component has two or more slots, allowing it to accommodate two or more wedges. In certain embodiments, a central component holds two wedges, and in certain embodiments, a central component holds three wedges. In certain embodiments, a central component includes a central channel allowing delivery of graft material through the channel. In certain embodiments, the central component and wedge are made of a material suitable for orthopedic surgery, including, but not limited to titanium, polyetheretherketone (PEEK), carbon fiber, ceramic, stainless steel or other materials commonly utilized within orthopedic implants, or combinations thereof.

In certain embodiments, the assemblable interbody cage 1850 comprises two or more wedges 1851, as shown in FIG. 48. In certain embodiments, two or more wedges 1851 are placed into position by being guided by a central component 1852. Referring to FIG. 49A-C, a central component 1852 has a proximal end 1853 and a distal end 1854. A distal end 1854 has a tip 1855, where in certain embodiments, a tip includes a feature for a gradual, atraumatic opening of tissue. In certain embodiments, the feature includes, but is not limited to, for example, a frustoconical shape, a bullet-nose shape, and a taper. In certain embodiments, a slot 1859 meets with a portion of the tip, and acts as a stop to prevent movement of a wedge. In certain embodiments, a central component 1852 includes a plurality of rails 1856. In certain embodiments, rails 1856 are positioned in a radially outward direction from the central component central stem 1857. A slot 1859 is formed in a space between the rails 1856. In certain embodiments, a slot 1859 has an opening 1860 connected with a proximal end of the central component. A rail 1856 further includes a retaining ledge 1861 in certain embodiments. In certain embodiments, a central component 1852 has a diameter 1862 that is adapted for use in an OLLIF approach. In certain embodiments, the diameter 1862 is approximately 9 mm, although it will be appreciated that the diameter can ranges from 3 mm to 15 mm in certain embodiments, and is of any size in certain embodiments. A stem 1866 attached to the central component central stem 1857. In certain embodiments, a central component includes an attachment hole 1858 located on a central component proximal end 1853, where a stem 1866 can attach to the central component. In certain embodiments, attachment of a central component to a stem is through a threaded connection.

Certain embodiments of the invention include two or more wedges 1851. Referring to FIG. 50A-D, a wedge 1851 has a proximal end 1864 and a distal end 1863 and oriented along a generally longitudinal axis 1874. In certain embodiments, a distal end 1863 has a ramped surface 1871, where a ramped surface helps to wedge a wedge into the disc space. A wedge 1851 has a rail cutout 1865 that accommodates an outer shape of a rail 1856. A wedge 1851 further includes a keyed element 1873 on the interior portion 1868 of the wedge 1851, where the keyed element 1873 runs substantially along a longitudinal axis 1874. The keyed element 1873 further includes a stem cutout 1867 in certain embodiments. Referring to FIG. 50E-F, in certain embodiments, a wedge 1877 has a distal end 1863, a proximal end 1864, an interior portion 1868, and an exterior portion 1869. It will be appreciated that in certain embodiments, the exterior portion of a wedge is available in a number of different shapes, included having a rounded surface or a planar surface. In certain embodiments, a wedge 1877 has a keyed element 1878 that is rounded. It is contemplated that in certain embodiments, a keyed element 1878 of a wedge 1877 fits through a track 1880 of a central component 1879 shown in FIG. 49D.

Referring to FIG. 51 showing a distal end perspective view of a plurality of wedges 1851, when properly assembled, a cavity 1872 is created among the wedge 1851 pieces. Referring to FIG. 52, a plurality of wedges 1851 are placed around a central component 1852, such that a central component 1852 is disposed between a cavity 1872 as shown in FIG. 51. In certain embodiments, the keyed element 1873 of a wedge 1851 is placed within a slot 1859 of the central component 1852. Referring to FIGS. 49B and 52, the retaining ledge 1861 of the rail 1856 constricts the keyed element 1873 of a wedge 1851 to a movement that is generally along a longitudinal axis.

In certain embodiments, the two or more wedges 1851 are sequentially delivered to a vertebral disc space. Referring to FIGS. 53A-D, the wedges are placed through a working sheath. An exemplary view through a working sheath boundary 1875, where the implant is viewed from the proximal side is shown in FIG. 53A-D. Referring to FIG. 53A, a first wedge 1851a is placed through the sheath boundary 1875, and positioned so that the keyed element 1873 fits between a first track 1856a and a second track 1856b. Referring to FIGS. 50B and 53A, a wedge has a curved surface 1870 located on an exterior portion 1869. Referring to FIG. 53A, the curved surface 1870 has a curvature that matches the curved surface of the working sheath boundary 1875. Furthermore, still referring to FIG. 53A, the stem 1866 has an edge that engages with a stem cutout 1867 located on the wedge 1851a. Initially, the central component, which is attached to a stem, is passed through a working sheath 1876. Once the central component is in position, one or more wedges are sequentially placed through the working sheath. As the wedge 1851a is passed through a working sheath 1876, the stem cutout 1867 and the curved surface 1870 of the wedge 1851a glide along the stem and the working sheath. The wedge is pushed out of the working sheath, until the wedge reaches the appropriate quadrant of a central component 1852. The wedge is further pushed until it is engaged with the central component. Referring to FIGS. 53A-D, once a first wedge 1851a is positioned into a central component 1852, the sheath is repositioned in order to insert the other wedges 1851b, c, d.

In certain embodiments, the stem 1866 has a non-circular profile. In certain embodiments, a stem 1866 has a square cross section. In certain embodiments, the stem 1866 generally has a non-circular profile to allow guidance of a wedge through the working sheath. In certain embodiments, a stem includes a cross section with other shapes. It will be appreciated that in certain embodiments, a central component has two or more tracks, allowing it to accommodate two or more wedges. In certain embodiments, a central component holds two wedges, and in certain embodiments, a central component holds three wedges. In certain embodiments, a central component includes a central channel allowing delivery of graft material through the channel. In certain embodiments, the central component and wedge are made of a material suitable for orthopedic surgery, including, but not limited to titanium, polyetheretherketone (PEEK), carbon fiber, ceramic, stainless steel or other materials commonly utilized within orthopedic implants, or combinations thereof.

The following paragraphs describe a preferred method of use of certain embodiments of the invention. One skilled in the art will recognize the variability in these steps based on factors such as surgeon preference and patient anatomy.

In certain embodiments, the method of use for the embodiments described herein are performed as shown in the flowchart of FIG. 33. In certain embodiments, the method includes identification of the route of entry step 1400. In certain embodiments, during the identification step 1400 the most appropriate route of entry is identified. In certain embodiments, a surgeon identifies the end point of the surgical approach by identifying the interbody space between the two vertebral bodies to be fused. One skilled in the art will appreciate the variability inherent in this step, depending on the intended target. This step will generally involve identifying the target point within an interbody space or on a vertebral body and determining the appropriate incision site. This step is often executed with the aid of imaging technology, such as Computerized Tomography (CT) scanning and/or biplanar fluoroscopy. In certain embodiments, an endoscope may be utilized in association with instrumentation for purposes associated with the inspection of the foramen and other structures near the passage prior to and following the insertion of instrumentation during the identification of the route of entry step 1400.

In certain embodiments, in order to accomplish the identification of the route of entry step 1400, the surgical team must first accomplish the positioning and confirming step. To do so, the patient to be subjected to the surgery utilizing the system described herein is first positioned on an operating table in a generally prone position. Typically, bi-planar C-Arm system is used for intra-operative fluoroscopic monitoring, and is used to confirm that the positioning of the patient's spine best resembles the neutral position, such that the unique anatomy and pathologies of the patient allow for a neutral position. As one skilled in the art would recognize, the term “neutral position” refers to a position that exhibits the three natural curves present in a healthy spine from a lateral view, wherein the cervical (neck) region of the spine (C1-C7) is bent inward, the thoracic (upper back) region (T1-T12) bends outward, and the lumbar (lower back) region (L1-L5) bends inward. In a substantially neutral position, the patient's spine will ideally show equal spacing between pedicles on an anterior-posterior fluoroscopic view, and superimposed pedicles on a lateral fluoroscopic view. Thus, in association with the positioning and confirming step, a surgeon will confirm that the patient is an appropriate candidate for fusion utilizing an OLLIF approach or determine an adequate explanation for why an OLLIF approach is inappropriate based on the patient's unique anatomy.

In certain embodiments, in association with the identification of the route of entry step 1400, the person performing the procedure performs a locating step. To perform the locating step, in an anterior-posterior view, the person performing the procedure locates the center of the disc via fluoroscopy in the vertical and horizontal planes. The surgeon or an assistant designates the midline and transverse plane by placing a radiopaque trajectory planning instrument over the skin while utilizing fluoroscopy. The person performing the surgery then engages in a step to mark a patient's skin to target the center of the disc. In certain embodiments the marks may include, for example, writing on a patient's skin. On a lateral plane, the radiopaque trajectory planning instrument determines the targeted disc's inclination angle. Following this, the person performing the surgery performs marking, whereby a skin marker is used to draw a line following the disc inclination angle (referred to as the “disc inclination line”) along the side of the patient towards the patient's posterior midline. In certain embodiments, the disc inclination line may indicate a trajectory that passes through the ilium, the sacrum, both or neither. On a lateral view, the person performing the surgery locates the center of the disc by repositioning the radiopaque trajectory planning instrument and drawing a second line along its trajectory on the skin's surface. Ideally, this second line will travel perpendicular to and intersect the disc inclination line. The person performing the procedure then engages in measuring to create a first depth measurement made along the disc inclination line from the dorsal skin to the center of the disc. The distance determined from this first measurement should then be applied from the midline marker laterally along the transverse plane distal from the center of the disc where a mark is made parallel to the midline. The intersection of this mark and the disc inclination line indicates the point of incision, or route of entry.

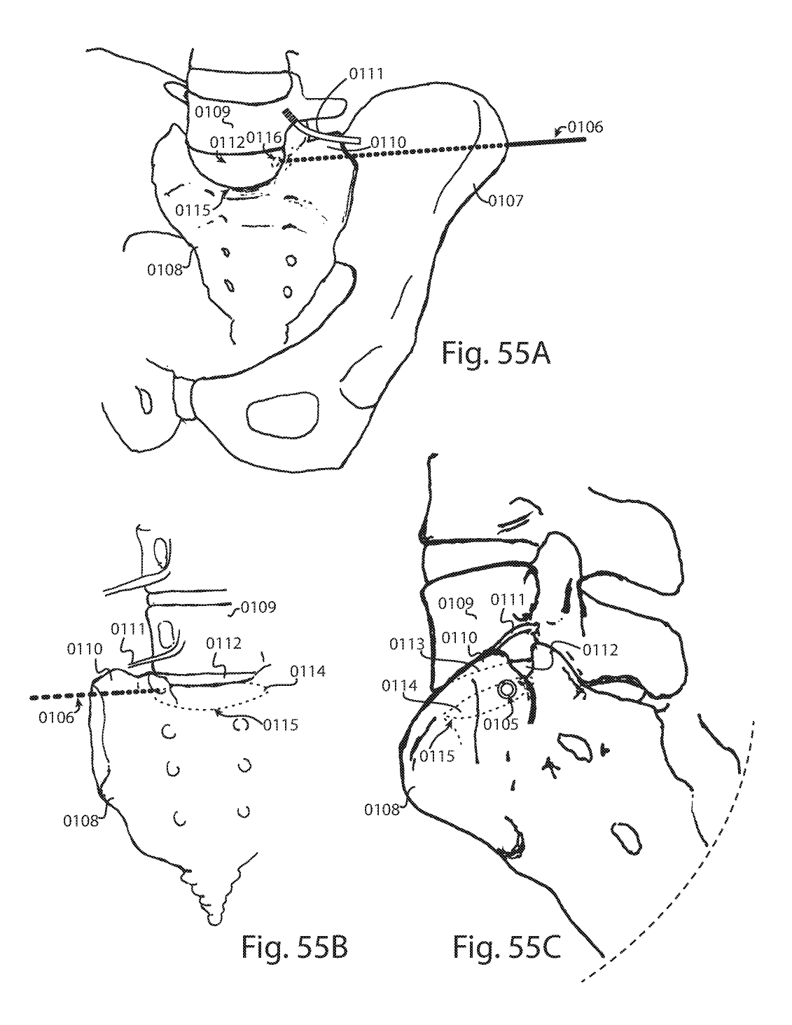

In certain embodiments of the invention, a passage 0106 is used to access the L5-S1 vertebral disc space. In certain embodiments, a passage 0106 traverses through both the sacrum 0108 and the ilium 0107, as depicted by FIG. 55A. In such embodiments, the passage 0106 through the ilium 0107 follows an oblique lateral route into the L5-S1 interbody space. In certain embodiments, the passage 0106 is located more posterior than the direct lateral route into the L5-S1 interbody space. The present inventors recognize that in certain embodiments, the passage 0106 along an oblique lateral trajectory is preferable to a direct lateral trajectory for accessing an L5-S1 vertebral disc space, as previously described direct lateral trajectories that use a monolithic, non-expandable cages are typically inferior, as the trajectory and type of implant used can lead to damage and intractable pain. In certain embodiments, a sheath that follows the passage 0106 has an outer diameter of no greater than 12 millimeters. Unlike the previously known direct lateral passage that passes solely through the ilium, certain embodiments use an oblique lateral passage 0106, as depicted in FIG. 55A-C, particularly using a sheath 0105 having an outer diameter of less than 12 millimeters, which leads to less pain for the patient following surgery. In certain embodiments, the present inventors have recognized that a passage 0106 that passes through both the ilium 0107 and through the sacral ala 0110, using a sheath 0105 having an outer diameter of less than 12 millimeters, leads to a reduction in pain for the patient following surgery. The present inventors have also recognized that a less desirable trajectory that is located above or through a portion of a sacral ala may lead to unintended deflection of instrumentation, including deflection caused by contact of instrumentation with the external surface of the sacral ala, superiorly and possibly into the L5 nerve root. Therefore, in certain embodiments of the invention, the passage passes through bone, and particularly through the sacral ala and ilium. The present inventors have recognized that in certain embodiments, a passage created to access the L5-S1 level using this approach traverses both the ilium and sacral ala, as the passage through bone enables the surgeon to avoid a trajectory that undesirably comes near or into contact with one or more nerves forming the boundaries of Kambin's Triangle.

In a certain embodiment, the sheath 0105 follows a passage 0106 through the ilium 0107. The sheath is angled such that it passes from the skin through both a posterior and superior quadrant of the ilium 0107 and the sacral ala 0110, and into the disc space 0112 adjacent and inferior to the L5 vertebral body 0109. Referring to FIG. 55A-C, it will be appreciated that the plane of the S1 superior endplate 0114, which is inferior to the L5-S1 disc space, angles inferiorly in an anterior direction relative to the plane of the endplate located superior to the L5-S1 disc space. For example, as shown in FIG. 55B-C, an approximate location of an S1 superior endplate 0114 is marked. Still referring to FIG. 55C, the approximate location of an edge 0113 of a L5 inferior endplate is marked. Referring to FIG. 55A-C, an anterior edge 0115 of the S1 superior endplate 0114 is angled inferiorly from a posterior edge of the endplate 0114. Previously described trajectories are located above or through a portion of a sacral ala, which may lead to unintended deflection of instrumentation in a superior direction, and possibly into the L5 nerve root. On the other hand, in certain embodiments, a passage 0106 passes through the sacral ala 0110 and forms an access opening 0116 within the L5-S1 disc space 0112. Once inside the bone, the passage 0106 is passed through the bone structures of the sacrum 0108 and ilium 0107 until the passage 0106 reaches the L5-S1 disc space 0112. Certain embodiments of the invention, as shown in FIG. 55A-C include a passage 0106 that is substantially lateral and generally stays within bone until it reaches a portion of the L5-S1 disc space 0112. In certain embodiments, the passage 0106 avoids potential damage to the L5 exiting nerve root 0111.

In certain embodiments, the identifying the route of entry step 1400 defines a path through both the ilium 0107 and the sacral ala 0110. In an embodiment, the identifying the route of entry step 1400 may involve tapping, drilling or otherwise passing a wire through both the ilium 0107 and the sacral ala 0110. In such embodiment, the guide wire may incorporate a drill trip configured to drill through both the ilium 0109 and the sacral ala 0110. In such embodiment, the present inventors intend for the surgeon to utilize a guide wire to define a path into the lower half of Kambin's Triangle, or the half of Kambin's Triangle located farthest away from the L5 nerve root, after passing through both the ilium 0109 and the sacral ala 0110. In such embodiment, the widen the passage 1403 step may include the utilization of drilling and/or boring instruments to drill and/or bore through the ilium and the sacrum. In certain embodiments, the passage 0106 traverses through at least part of the area within Kambin's Triangle 0104.

In certain embodiments, the method of use for the embodiments described herein includes an insert needle 1401 step. One skilled in the art will appreciate the variability inherent in this step, depending on the intended target. In association with this step, prior to making an incision, local anesthetics may be used at the point of incision. Generally, in association with this step, a 9-12 mm incision is made at the point of incision. In the method associated with the preferred embodiment, a surgeon will insert a neuromonitoring probe, for example, a unidirectional, monopolar neuromonitoring probe, through the incision to target an interbody space through Kambin's Triangle. During the insert needle 1401 step, in the preferred embodiment, a surgeon should pass between the structures comprising Kambin's Triangle 1402. In an embodiment, the neuromonitoring probe has a slot either on the lateral surface, or centered within that spans the length of the probe to hold a slidably and removably engaged trephine needle, also known as Kirschner Wire or K-Wire. In certain embodiments, neuromonitoring is performed with the instrument described for FIGS. 39A-I. Using the neuromonitoring probe, an exiting nerve root 0102, which forms the hypotenuse of Kambin's Triangle is mapped and identified. Surgeon should ensure that the neuromonitoring probe trajectory passes through Kambin's Triangle. Kambin's Triangle is an area that may be conceptualized as substantially a right triangle that is defined by the exiting nerve—which forms the hypotenuse—the superior endplate of the caudal vertebral body 0101—which forms the base—and the traversing nerve 0102—which forms the height. Those skilled in the art recognize that Kambin's Triangle may not form the precise shape of a triangle. Such mapping and identification takes place via electrical stimulation of the associated nerve structures. One skilled in the art will recognize this standard surgical practice as Triggered EMG. The surgeon determines nerve depolarization, for example, at a minimum level of 3 mA, to establish safe distance from the nerves associated with Kambin's Triangle. Anterior-posterior and lateral fluoroscopic imaging is viewed to confirm that the neuromonitoring probe is placed through Kambin's Triangle and touching the substantially lateral aspect of the targeted interbody space. Once safe placement and safe trajectory is confirmed, more specifically by confirmation of the trajectory through the “Safe Zone” of Kambin's Triangle, variably defined as the “lower half of Kambin's Triangle” or the “half of the area between the structures forming the boundary of Kambin's Triangle farthest away from the exiting nerve root,” the trephine needle is then be placed into the annulus of the targeted disc via the previously described slot. The neuromonitoring probe is then removed leaving the trephine needle to maintain and identify the safe trajectory though Kambin's Triangle to the interbody space.

In certain embodiments, the neuromonitoring probe is incorporated into the first dilator, generally through use of a neuromonitoring instrument, as depicted in FIGS. 39A-I. In certain embodiments, the user places a standard disposable monopolar probe within a sheath or a first dilator, until the distal end of the monopolar probe makes contact with the stainless steel distal end of the first dilator. The user then bends the shaft of the standard disposable monopolar probe at an angle of approximately 30 degrees within the slot of the first dilator. The user then attaches a quarter-inch square quick connect palm handle to the quick connect feature of the first dilator. The user then slides the sheath onto the body of the first dilator and engages the pin features to accomplish a fully assembled state. The user then delivers the fully assembled first dilator into the body at the previously determined trajectory. As the user delivers the fully assembled neuromonitoring instrument to the targeted interbody space, the user views fluoroscopic images to determine when the distal tip of the first dilator contacts the annulus of the targeted interbody space. In certain embodiments, the user then stimulates the standard disposable monopolar probe to thereby stimulate the stainless steel distal end of the first dilator. The user then monitors the neuromonitoring threshold, and if the threshold is satisfactory, the user then impacts the palm handle at the proximal end of the first dilator with a mallet to dock the distal end, including, for example, the flattened tip and the conical tip into the disc space. The user impacts the handle until the opening of the sheath is fully docked within the disc space, as observable by viewing fluoroscopic imaging. The user then rotates the first dilator to disengage the pins from the sheath impact collar. The user then removes the first dilator and the standard disposable monopolar probe leaving only the sheath in place.

Still referring to FIG. 33, the method of use for the embodiments described herein includes a step 1403 to widen the passage. This step encompasses the insertion of one or more cannulas over a trephine needle placed into the body in the previous step in sequential order, creating a wider channel. First, in the method associated with certain embodiments, an initial dilator instrument, also referred to as a dilator or a first dilator is inserted over a trephine needle to widen an opening. The first dilator 1500 is passed over a trephine needle with initial reference marking 1503 facing parallel to the direction of an exiting nerve root 0102, as determined from previous nerve mapping and anatomical knowledge. In varying embodiments, where the trephine needle and/or the first dilator is incorporated into the first dilator, all or part of the step 1403 to widen the passage and the insert needle step 1401 may be combined. It will be appreciated that in certain embodiments, an initial dilator instrument, also referred to as a first dilator, has features to widen the path without requiring a trephine needle.

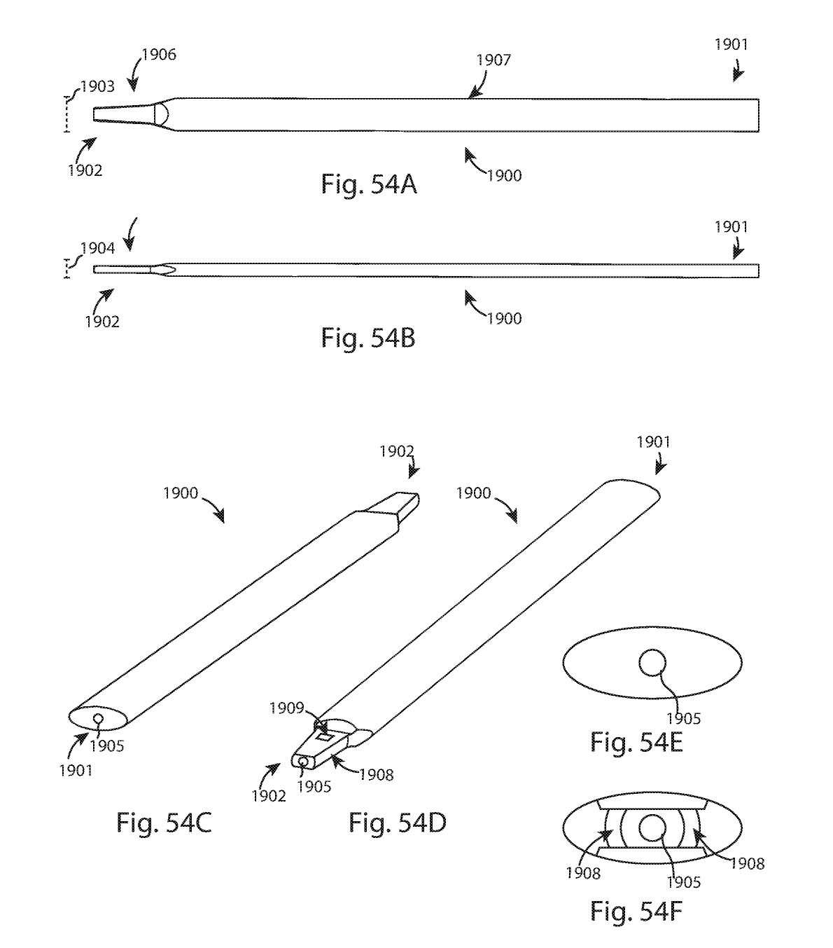

In certain embodiments, the first dilator, once positioned safely through Kambin's Triangle, is rotated 90 degrees along a trephine needle. This rotation effectively displaces a traversing nerve root 0102 away from the trajectory of the approach into the interbody space. Referring to FIGS. 54A-F, in certain embodiments, a dilator 1900 has a substantially elongate form. A dilator 1900 includes a proximal end 1901 and a distal end 1902. Referring to FIGS. 54A-B, in certain embodiments, a dilator 1900 has a first dimension 1903 that is greater than a second dimension 1904. A profile of a dilator shaft 1907 has a shape that is generally elliptical, as shown in FIGS. 54E-F. In certain embodiments, a dilator 1900 includes a cannula 1905 connecting the proximal end 1901 and a distal end 1902. In certain embodiments, the distal end has a narrowed tip 1906. Generally, the overall shape of the dilator 1900 allows positioning the dilator into Kambin's Triangle, and rotating it to displace a nerve root. In certain embodiments, the narrowed tip includes a taper that allows penetration into a vertebral disc. In certain embodiments, a side wall 1908 of a narrowed tip 1906 has a curvature that facilitates turning the dilator while the tip is in the disc space. In certain embodiments, a dilator 1900 can be used as a first dilator or an initial dilator during the approach as described herein. It will be appreciated that certain embodiments of a dilator 1900 include a reference marking 1909. A reference marking includes, for example, a radiopaque marker, a radiolucent marker, a protrusion, a divot, or other physical feature that allows a surgeon to observe the orientation of an instrument.

In certain embodiments, the second dilator 1508 is then advanced over the first dilator 1500 of dilator 1900 through Kambin's Triangle to the substantially lateral aspect of the disc. In an embodiment, the second dilator 1508 is advanced over the first dilator 1500 with initial reference marking 1503 facing toward an exiting nerve root 0102 of Kambin's Triangle.

In certain embodiments, a third and optionally a fourth dilator may be used in addition to further expand the path of approach to an interbody space, preceding the placement of the final dilator instrument or sheath 1514. In certain embodiments a sheath that has a profile that is substantially similar to the profile of a dilator shaft 1907, for example, an elliptical profile.

In certain embodiments, a sheath 1514 is positioned over the first dilator 1500 or a second dilator 1508. An impactor device 1528 is optionally used to seat a sheath 1514 into an interbody space. In certain embodiments, an impactor device 1528 includes a through opening 1529 that accommodates, for example, a guide wire. An impactor device 1528, in certain embodiments, is shown in FIG. 12.

In a certain embodiment, once sheath 1514 is placed and anchored between vertebral endplates, a safe passage is established through a patient's superficial soft tissue, between the structures comprising Kambin's Triangle, and into an interbody space. In varying embodiments, the K-Wire, first dilator 1500, and second dilator 1508 if previously placed are removed, leaving only sheath 1514 in place.

In certain embodiments, the disc is prepared for a placement of an implant. During a disc preparation step 1404, steps associated with a discectomy and annulotomy are performed. In certain embodiments, discectomy instrumentation is used in steps related to discectomy and annulotomy. In an embodiment, the person performing the surgery removes interbody disc material using discectomy instrumentation to cut through the nucleus of a disc. Subsequently, the person performing the surgery then utilizes the discectomy instrumentation to remove the disc material through the sheath 1514. In certain embodiments, the discectomy instrumentation also prepares the superior and inferior endplates of an interbody space, causing bleeding of such endplates. In certain embodiments, an endoscope may be utilized in association with discectomy instrumentation for purposes associated with the visual inspection of the discectomy and endplate preparation prior to and following the insertion of discectomy instrumentation.

In certain embodiments, an implant trialing step is optionally performed after removing disc material. In certain embodiments, trialing determines the appropriate size of expandable interbody cage 1000 to be placed into the interbody space. A trialing instrument is placed through the sheath 1514 and into an interbody space. In certain embodiments, trialing instrument is performed with an expandable cage similar to those shown in FIGS. 14-32, and similar to those shown in FIGS. 40-44. In certain embodiments, a delivery tool or instrument is used to deliver a trial instrument to the disc space. In certain embodiments, a deliver tool or instrument described for FIGS. 45A-E is used. Certain embodiments of a trialing instrument incorporates a handle, which, when squeezed, distracts an interbody space. Once the desired amount of distraction is achieved, the person performing the procedures engages in selecting an expandable interbody cage 1000 with appropriate height dimensions in its deployed configuration to match the distraction achieved with a trialing instrument.

In certain embodiments, following the trialing step, the person performing the surgery performs the step to inserting an implant or cage. During the insert cage step or deliver apparatus step 1405, one or more than one implant is placed into the interbody space by passing through a sheath. In certain embodiments of the deliver apparatus step 1405, a non-expandable cage or implant is inserted into an interbody space by passing through a sheath. In certain embodiments, during the insert cage step or deliver apparatus step 1405, an expandable interbody cage 1000 is placed into an interbody space by passing through a sheath. The person performing the surgery then utilizes a deployment tool to transform the implant from transit configuration or a retracted configuration to a deployed configuration or an expanded configuration. Once the expandable interbody cage 1000 is placed and expanded, the person performing the procedure then may confirm or verify 1407 appropriate placement utilizing with fluoroscopic imaging. Following confirmation of expandable interbody cage placement location, any remaining instrumentation including the sheath 1514 may be removed 1408. The person performing the procedure may then engage in the standard surgical close of the passageway.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. For the purposes of illustration related to example embodiments disclosed herein, “distal” is defined as the direction away from the surgeon, and “proximal” is defined as the direction toward the surgeon. The terms “a” and “an” are defined as one or more unless explicitly stated otherwise herein. The terms “substantially”, “essentially”, “approximately”, “about” or any other version thereof, are defined as being close to as understood by one of ordinary skill in the art. The terms “coupled” and “linked” as used herein is defined as connected, although not necessarily directly and not necessarily mechanically. A device or structure that is “configured” in a certain way is configured in at least that way, but may also be configured in ways that are not listed. Also, the sequence of steps in a flow diagram or elements in the claims, even when preceded by a letter does not imply or require that sequence.

What is claimed is:

1. A surgical method for fusing vertebra, comprising:
identifying a route into an interbody space on a trajectory that passes through the structures of Kambin's Triangle;
widening the passage;
placing a sheath through the passage;
removing disc material through the sheath;
transiting an implant through the sheath; and
placing the implant within the interbody space prior to removing said sheath.

2. The method of claim 1, further comprising inserting a trephine needle through said trajectory after identifying said route through a safe zone of Kambin's Triangle.

3. The method of claim 1, wherein transiting said sheath with said implant comprises inserting an expandable interbody cage in a retracted configuration, said expandable interbody cage comprising a form following a longitudinal axis and defining a proximal end, and a distal end;
said expandable interbody cage further comprising a proximal end link, a distal end link, and a center link;
said proximal end link hingeably connected with said center link; and
said distal end link hingeably connected with said center link;
wherein pulling said distal end link towards said proximal end link pushes said center link away from said longitudinal axis.

4. The method of claim 1, wherein transiting said sheath with said implant comprises inserting an expandable interbody cage in a retracted configuration, said expandable interbody cage comprising a form following a longitudinal axis and defining a proximal end, and a distal end;
said expandable interbody cage further comprising a proximal element, a proximal end link, a distal element, a distal end link, and a center link;
said proximal element hingeably connected with said proximal end link;
said proximal end link hingeably connected with said center link;
said distal element hingeably connected with said distal end link; and
said distal end link hingeably connected with said center link;
wherein pulling said distal element towards said proximal element pushes said center link away from said longitudinal axis.

5. The method of claim 1, wherein transiting said sheath with said implant comprises inserting an assemblable interbody cage, said assemblable interbody cage comprising a form following a longitudinal axis and defining a proximal end, and a distal end;
said assemblable interbody cage further comprising a central component and a wedge; said central component comprising a distal end, a proximal end, a stem, and at least two rails; a tip located at said central component distal end;
the at least two rails positioned in a substantially radial orientation from said central component stem; the space between at least two rails defining a slot;
said wedge comprising a proximal end, a distal end, and defining an exterior surface and an interior surface, the wedge comprising a keyed element on said interior surface, wherein the keyed element of said wedge is slideable along the slot of the central component; and
wherein the central component is inserted through the sheath before the wedge.

6. The method of claim 1, wherein the expanding step is accomplished using an inserter.

7. The method of claim 1, wherein the step of widening the passage comprises inserting a first dilator, said first dilator compromising a distal end, a proximal end, and a cannula; said cannula connected with an opening on the dilator proximal end and extending towards the dilator distal end; and the distal end of said first dilator comprising a taper.

8. The method in claim 7, wherein the steps of widening the passage and placing the sheath further comprises assembling the sheath with the first dilator.

9. The method of claim 1, wherein the step of widening the passage comprises creating an aperture through at least one of an ilium and a sacrum.

10. The method of claim 10, wherein the step of identifying the route of entry comprises passing through the ilium and the sacrum to an L5-S1 interbody space.

11. A system for a sheathed oblique lateral interbody fusion procedure, comprising:
an expandable interbody cage;
an inserter configured to expand said expandable interbody cage;
a first dilator; and
a sheath.

12. The system of claim 11, wherein said first dilator comprises a distal end, a proximal end, and a cannula; said cannula connected with an opening on the dilator proximal end and extending towards the dilator distal end; and the distal end of said first dilator comprising a taper.

13. The system of claim 12, wherein said first dilator cannula is connected with an aperture located towards the dilator distal end; and wherein said dilator distal end further comprises a flattened tip.

14. The system of claim 12, wherein said first dilator proximal end further comprises a slot adapted to receive a neuromonitoring probe.

15. The system of claim 11, further comprising discectomy instrumentation.

16. The system of claim 15, wherein said discectomy instrumentation further comprises a cutter assembly.

17. The system of claim 15, wherein said discectomy instrumentation further comprises a tissue extractor.

18. The system of claim 11, wherein said expandable interbody cage comprises a form following a longitudinal axis, and defining a proximal end, and a distal end;
said expandable interbody cage further comprises a proximal end link, a distal end link, and a center link;
said proximal end link hingeably connected with said center link; and
said distal end link hingeably connected with said center link;
wherein pulling said distal end link towards said proximal end link pushes said center link away from said longitudinal axis.

19. The system of claim 11, wherein said expandable interbody cage further comprises a proximal element, a proximal end link, a distal element, a distal end link, and a center link;
said proximal element hingeably connected with said proximal end link;
said proximal end link hingeably connected with said center link;
said distal element hingeably connected with said distal end link; and
said distal end link hingeably connected with said center link;
wherein pulling said distal element towards said proximal element pushes said center link away from said longitudinal axis.

20. The system of claim 11, wherein said expandable interbody cage comprises a form following a longitudinal axis and defining a proximal end, and a distal end;
said expandable interbody cage further comprising a central component and a wedge; said central component comprising a distal end, a proximal end, a stem, and at least two rails; a tip located at said central component distal end;
the at least two rails positioned in a substantially radial orientation from said central component stem; the space between the at least two rails defining a slot; and
said wedge comprising a proximal end, a distal end, and defining an exterior surface and an interior surface, the wedge comprising a keyed element on said interior surface; wherein the keyed element of said wedge is slideable along the slot of the central component.

21. The system of claim 11, wherein said sheath comprises an outer diameter no greater than 12 mm.
